SEQUENCE LISTING (6 sequences)

SEQ ID NO: 1 - DNA, 24 bp, Artificial Sequence
  Primer
cttgaagacttgaattagattaca

SEQ ID NO: 2 - DNA, 25 bp, Artificial Sequence
  Primer
tgttgtagccaaaattggaaagagc

SEQ ID NO: 3 - DNA, 23 bp, Artificial Sequence
  Primer
tggtgctccatgaggagaaccgc

SEQ ID NO: 4 - DNA, 22 bp, Artificial Sequence
  Primer
gccaggagcctgcctcttccac

SEQ ID NO: 5 - DNA, 26 bp, Artificial Sequence
  Primer
tgtggctaccctgccaagcgcaagag

SEQ ID NO: 6 - DNA, 20 bp, Artificial Sequence
  Primer
acagctgccctcttgggttt

(19) United States
(12) Patent Application Publication
(10) Pub. No.: US 2024/0033317 A1
(43) Pub. Date: Feb. 1, 2024

(54) USES OF HYPOXIA-INDUCIBLE FACTOR INHIBITORS FOR TREATING TP53-MUTATED ACUTE MYELOID LEUKEMIA

(71) Applicant: Children's National Medical Center, Washington, DC (US)

(72) Inventors: Yang LIU, Potomac, MD (US); Yin WANG, Washington, DC (US); Yan LIU, Washington, DC (US); Christopher BAILEY, Merritt Island, FL (US)

(21) Appl. No.: 17/904,142

(22) PCT Filed: Feb. 12, 2021
(86) PCT No.: PCT/US2021/017838

§ 371 Date: Aug. 12, 2022

(51) Int. Cl. A61K 38/08; A61K 9/127; A61P 35/02

(52) U.S. Cl. CPC A61K 38/08; A61K 9/1271; A61P 35/02

(57) ABSTRACT

The present invention relates to treating TP53-mutated AML using a Hypoxia-Inducible Factor (HIF inhibitor). The invention further relates to a new HIF inhibitor formulation with longer half-life and significantly improved therapeutic effect for TP53-mutated AML.

### FEDERALLY SPONSORED RESEARCH OR DEVELOPMENT

This invention was made in part with Government support under Grant Nos. CA171972, CA183030, and CA164469 awarded by the National Institutes of Health National Cancer Institute. The Government has certain rights in this invention.

### FIELD OF THE INVENTION

The present invention relates to hypoxia-inducible factor inhibitors, pharmaceutical compositions thereof, and use of the foregoing for treating TP53-mutated Acute Myeloid Leukemia.

### BACKGROUND OF THE INVENTION

Somatic TP53 mutations are frequently detected in a variety of cancers, with different frequencies dependent on the cancer type. TP53 mutations are found in acute myeloid leukemia (AML) patients with a frequency of over 10%, especially in cases with complex karyotypes, and are found at even higher frequencies in therapy-related AML (between 20-40%). Overall, TP53 mutations are associated with very poor prognosis, with poor responses to chemotherapy and allogeneic stem cell transplantation. Response rates to hypomethylating agents are higher, but responses are not durable. Restoration of p53 function is a possible strategy to suppress cancer growth, but no targeted therapy is available clinically to restore p53 function. It is of great interest to test whether other pathways activated by TP53 mutations can be therapeutically targeted.

Accumulation of HIF-1α associated with loss of p53 function has been associated with an unfavorable prognosis and increased risk of patient mortality in some malignancies. It has been shown that, even under normoxia, HIF1α signaling was selectively activated in the stem cells of murine T-ALL and human AML. Other studies have confirmed that the pathway is also critical for the maintenance of chronic myeloid leukemia stem cells and could be targeted to provide therapeutic effect. Importantly, the HIF1α inhibitor echinomycin efficiently eradicated murine leukemia and was highly effective and selective in eliminating AML stem cells without adverse effect on normal hematopoietic stem cells. Although TP53 mutations are observed in approximately 10% of AML samples and are associated with devastating prognosis, it has not been previously documented whether inhibition of HIF-1α accumulation in TP53-mutated AML suppresses leukemia cell progression. Therefore, it is of great interest to test whether targeting the HIF-1α pathway can provide a therapeutically effective strategy for TP53-mutated AML.

### SUMMARY OF THE INVENTION

Provided herein is a method for treating Acute Myeloid Leukemia (AML) in a mammal in need thereof, which may comprise administering a Hypoxia-Inducible Factor (HIF) inhibitor to the mammal. Also provided herein are use of the HIF inhibitor in the manufacture of a medicament for treating AML, and the HIF inhibitor for use in treating AML. The HIF inhibitor may be contained in a pharmaceutical composition or formulation described herein. The AML may be TP53-mutated AML. The mammal may be a human. The HIF inhibitor may be a HIF1α inhibitor, and may be echinomycin, 2-methoxyestradiol, or geldanamycin. The HIF inhibitor, which may be echinomycin, may be administered at a non-toxic dose, which may be 1-1000 μg/m².

The TP53-mutated AML may be characterized by enriched activity of one or more HIF1A target genes, as compared to one or more wild-type TP53 AML samples. The one or more HIF1A target genes may comprise a gene, which may be human, selected from the group consisting of TFRC, CMYC, HK1, SLC2A1, SNAIL, ALDOC, CP, TF, GLUT, and VEGF. The TP53-mutated AML may be refractory to standard therapy, which may comprise administering daunorubicin and cytarabine (DNR+Ara-C) to the mammal.

Also provided herein is a pharmaceutical composition comprising the HIF inhibitor formulated in PEGylated liposomes. The PEGylated liposomes may comprise one or more of hydrogenated soy phosphatidylcholine (HSPC), cholesterol, and distearoylphophatidylethanolamine (DSPE)-mPEG2000. The HSPC, cholesterol, and DSPE-mPEG2000 may be present in the PEGylated liposomes at molar ratios of 50-60%, 30-40%, and 1-5%, respectively. The PEGylated liposomes may comprise, as molar ratios, about 57% HSPC, about 38% cholesterol, and about 5% DSPE-mPEG2000. The pharmaceutical composition may comprise the HIF inhibitor and PEGylated liposomes at a molar ratio of 3% drug/lipid. The HIF inhibitor may be echinomycin. The pharmaceutical composition may be administered in a method described herein.

### BRIEF DESCRIPTION OF THE DRAWINGS

FIGS. 1A-G. Echinomycin significantly inhibits expansion of human TP53-mutant AML cells in vitro. FIG. 1A. HIF-1α target genes are highly expressed in TP53-mutated AML from patients. Gene Set Enrichment Analysis (GSEA) was performed to examine the expression differences of HIF-1α targets between patients with TP53-mutated AML and TP53-wild type AML from the public database. GSEA result showing significant enrichment (FDR<0.1) of the curated HIF-1α target genes and hallmark gene sets from MSigDB in TP53-mutated AML patients (32 cases) compared with patients with TP53-wild type AML (419 cases). In silico analysis of data reported by Tyner et al. FIG. 1B. Sensitivity to echinomycin of TP53-mutated AML cells from patients. AML cells were incubated for 24 hours in culture medium with echinomycin at increasing concentrations, ranging from 0.05 to 4.05 nM. After the incubation period, MTT metabolization of treated cells was measured by absorbance and is expressed as the mean±SD (n=3) of the percentage of the value of untreated cells. At the same time, 2×10⁴ treated cells were seeded in methylcellulose medium for colony-forming assay. Colonies were counted on day 7 to 10 in different AML cells. CFU is expressed as the mean±SD (n=3) of the percentage of the value of untreated cells. Data are shown for one experiment and are representative of two independent experiments. FIGS. 1C-F. Selectivity of echinomycin for CD34⁺ CD38⁻ subset of TP53-mutated AML cells. Cells from colonies as described in FIG. 1A were resuspended and stained with anti-human CD45, CD34, CD38, CD14, CD33, HLA-DR, CD11b and CD123, and analyzed by FACS. Representative FACS plots show the percentage of human CD34⁺ CD38⁻ cells (FIGS. 1C, E) or cells with leukemia markers (FIGS. 1D, F) in colonies treated with echinomycin. FIG. 1G. CD34⁺CD38⁻ AML subsets have higher HIF1α activity compared to bulk tumor cells. Two TP53-mutated (AML 277, AML 172) and two TP53-wild type (AML 71, AML 132) clinical AML samples were sorted into CD34⁺CD38⁻, CD34⁻ CD38⁻, CD34⁻CD38⁺, or CD34⁺CD38⁺ fractions by FACS and qPCR was subsequently performed to analyze mRNA levels of HIF-1α target genes among each subset. The data are summarized and statistical significance was determined by unpaired t test of the CD34⁺CD38⁻ cells vs the other subsets (* p<0.05; ** p<0.01; *** p<0.001; **** p<0.0001; ns, not significant). All data are representative of two independent experiments.

FIG. 2. HIF-1α target genes are highly expressed in TP53-mutated AML from patients. Mean value of HIF-1α target marker genes in AML patients from The Cancer Genome Atlas (TCGA). Statistical significance of the expression of HIF-1α target marker genes in TP53-mutated AML patients (8 cases) compared with TP53-wild type AML patients (162 cases) was determined by the Wilcoxon test.

FIGS. 3A-B. Sensitivity to Echinomycin of TP53-mutated primary AML cells. (FIGS. 3A,B) AML cells (FIG. 3A, PB-172 and FIG. 3B, PB-277) were incubated for 48 hours in culture media with increasing doses of Echinomycin ranging from 0.05 to 4.05 nM. After the incubation period, MTT metabolization of treated cells was measured by absorbance and is expressed as the mean±SD (n=3) of the percentage of the value of untreated cells. At the same time, 2×10⁴ treated cells were seeded in methylcellulose medium for colony forming assay. Colonies were counted at day 7 to 10 dependent on different AML cells. Cfu is expressed as the mean±SD (n=3) of the percentage of the value of untreated cells. Data is shown for one experiment and is representative of two independent experiments.

FIGS. 4A-F. Echinomycin significantly suppresses expansion of human TP53-mutated AML cells in a mouse xenograft model. FIG. 4A. Experimental design for the AML-147 xenograft model. NSG mice were irradiated with 1.3 Gy and transplanted with 1×10⁶ human TP53-mutated AML-147 cells by i.v. injection (day 0). Peripheral blood tests were performed before drug treatment on day 98, and after treatment on days 114, 124, and 155, indicated by red arrows. Drugs were administered by i.v. injection beginning on day 104. Individual doses are indicated by green arrows for echinomycin (50 μg/kg) or blue arrows for conventional therapy. The regimen for conventional therapy consisted of cytarabine (5 mg/kg)+daunorubicin (0.5 mg/kg) for 3 continuous days, then daunorubicin (0.5 mg/kg) for 2 continuous days. FIG. 4B. Human CD45⁺ cells in blood of AML-147 recipients described in FIG. 4A were detected by FACS analysis on day 98 after transplantation. Frequencies of human CD45⁺ in blood of recipients before treatment (Day 98) and after echinomycin treatment (Day 114, 124 and 155) are summarized. Statistical analysis was performed by one-way ANOVA with Tukey's multiple comparisons test, p values are shown for either drug treatment group vs vehicle control.

FIG. 4C. Kaplan-Meier survival curves of AML-147 recipients in FIG. 4A treated with echinomycin, DNR+Ara-C, or vehicle. Data are representative of two independent experiments.

FIGS. 4D-F. CD38⁻CD34⁺ subset from TP53-mutated AML cells are more sensitive to Echinomycin in vivo. Single cell suspensions isolated from spleens of mice described in FIG. 4A. were stained with anti-human CD45, CD34, CD38, CD14, CD33, HLA-DR, CD11b and CD123, and analyzed by FACS. Representative FACS plots showing the percentage of human CD38⁻CD34⁺ cells in splenocytes from mice treated with Echinomycin, DNR+Ara-C, and vehicle are shown in FIG. 4D and summarized in FIG. 4E, and representative FACS plots are shown for various leukemia marker expression patterns among the groups in FIG. 4F. N=3 mice per group for vehicle and DNR+Ara-C groups, n=5 for echinomycin group, and statistical significance was determined by unpaired t tests between groups (* p<0.05; ** p<0.01; *** p<**** p<0.0001; ns, not significant). All data are representative of two independent experiments.

FIGS. 5A-E. Echinomycin has therapeutic efficacy in most human TP53-mutated AMLs. FIG. 5A. NSG mice irradiated with 1.3 Gy were given i.v. injection of 1×10⁶ human primary TP53-mutated AML cells (AML-281). Human CD45⁺ cells in blood of recipients were detected by FACS analysis on day 66 after transplantation. Starting on day 67, mice received 10 μg/kg of echinomycin or vehicle by i.p. injections on a schedule consisting of 3 QDx5 cycles, each separated by 2 days rest. The FACS file showed the percentage of human CD45⁺ in blood of recipients before treatment (Day 66) and after echinomycin treatment (Day 232, 254 and 300) in two representative mice (vehicle and echinomycin treatment). FIG. 5B. Summary of percentages of human CD45⁺ in blood of all recipients treated with vehicle or echinomycin. Data are representative of two independent experiments. FIG. 5C. NSG mice were irradiated with 1.3 Gy and given 1×10⁶ human primary TP53-mutated AML cells (AML-227) by i.v. injection and were treated with echinomycin according to the schedule in A. Summary of percentages of human CD45⁺ in blood of all recipients treated with vehicle or echinomycin on day 300 is shown. Data are representative of two independent experiments. FIG. 5D. NSG mice were irradiated with 1.3 Gy and given i.v. injection of 1×10⁶ human TP53-mutated AML cells (AML-012). Twenty days later, mice received 50 μg/kg of echinomycin or vehicle by i.p. injection on a schedule consisting of Q2D×10. Kaplan-Meier survival curves of NSG recipients treated with echinomycin and vehicle are shown. Data are representative of two independent experiments. FIG. 5E. NSG mice were irradiated with 1.3 Gy and given i.v. injection of 2×10⁶ human TP53-wild type AML cells (AML-132). Ninety-three days later, the mice were treated with echinomycin (50 μg/kg) or vehicle, Q2D×10, by i.p. (n=5/group). Kaplan-Meier survival curves of NSG recipients treated with echinomycin and vehicle are shown. Data are representative of two independent experiments.

FIGS. 6A-D. Representative LC-MS/MS chromatograms of (FIG. 6A) a blank mouse plasma sample, (FIG. 6B) a blank mouse plasma sample spiked with internal standard (IS), (FIG. 6C) a blank plasma sample spiked with standard Echinomycin at the LLOQ level (0.05 ng/mL), and (FIG. 6D) a mouse plasma sample taken at 1 h after IV administration of Echinomycin at the dose of 100 ug/kg in a xenograft mouse model.

FIGS. 7A-G. Liposomal echinomycin suppressed the growth of human TP53 null THP1 cells and patient-derived xenograft TP53-mutated AML 12 cells in a xenograft mouse model. FIG. 7A. Pharmacokinetic study of echinomycin in plasma of mice. NSG mice were given a single i.v. injection of control vehicle or echinomycin at 100 ug/kg in one of three different formulations (Free-EM: echinomycin dissolved in DMSO then dispersed in PBS; CrEL-EM, echinomycin dispersed in cremophor; Lipo-EM, liposomal echinomycin). Blood samples were collected at 0.25, 0.5, 1, 2, 4, 8, 16 and 24 h after dosing. The concentration of echinomycin in plasma was analyzed by LC-MS/MS method. FIG. 7B. Echinomycin inhibits the growth of TP53-null THP1 cells. THP1 cells (1×10⁵) were seeded in RPMI 1640 culture medium in a 24-well plate and cultured for 24 hours. The cells were treated with echinomycin at different concentrations dissolved in DMSO (free echinomycin) for 48 hrs. MTT [3-(4,5-dimethylthiazol-2-yl)-2,5-diphenyltetrazolium bromide] (10 μl of 5 mg/ml) was added to each well containing THP1 cells. After 2 to 4 hours in culture, cells were centrifuged and the formazan crystals were resuspended in 150 μl DMSO and optical density was read at 490 nm. The values for the measured wells after background subtraction are summarized. Data shown are means and S.D. of triplicate wells and are representative of three independent experiments. FIG. 7C. Quantitative-PCR analysis of mRNA isolated THP1 cells cultured with vehicle or echinomycin (0.45 nM) for 24 hrs. Statistics are by paired student's t test for wells analyzed in triplicate per each group (* p<0.05; ** p<0.01; *** p<0.001; **** p<0.0001; ns, not significant). Data are representative of two independent experiments. FIG. 7D. Dosing regimen of echinomycin treatment for mice transplanted with luciferase-transduced THP1 cells. Day 0 indicates the date of birth and 1×10⁶ of THP1 cells are transplanted into pups via intrahepatic injection on day 2 (blue arrow). The baseline pre-treatment bioluminescence is determined by imaging the mice on day 5. Mice were treated with 10 μg/kg CrEL-EM or liposomal echinomycin, or vehicle, according to a Q2D×15 schedule starting on day 3 after tumor transplantation. FIG. 7E. Therapeutic effect of echinomycin. Serial imaging was performed for echinomycin- or vehicle-treated NSG recipients of THP1 as described in FIG. 7D. Imaging is shown for each group on day 5, corresponding to the pre-treatment values (before), and on days 3, 6, 9, 12 and 15 after doses. Data are representative of 3 independent experiments. FIG. 7F. Quantification of bioluminescence intensity of mice depicted in FIG. 7E. Bioluminescence intensity (photons/second) was measured and plotted before and after treatment and is shown as means±SEM (n=7 per group). Statistics are by t test (* p<0.05; ** p<0.01; *** p<0.001; **** p<ns, not significant). FIG. 7G. Kaplan-Meier survival curves are shown for the mice as described in FIG. 7D. Liposomal echinomycin had significantly prolonged survival compared with vehicle treatment. Data are representative of two independent experiments.

FIGS. 8A-G. Liposomal Echinomycin suppressed the growth of human patient-derived xenograft TP53-mutated AML cells in a xenograft mouse model. FIG. 8A. Liposomal Echinomycin, but not CrEL-EM, suppressed BM AML-012 blasts in the xenograft mouse model. NSG mice were transplanted with TP53-mutated AML-012 (twice passaged in NSG mice) via i.v. and treated with vehicle, CrEL-EM (0.1 mg/kg) or Lipo-EM (0.35 mg/kg) via i.v., once every three days for a total of 5 doses, beginning on day 10 after transplantation. The shorter survival times for all mice is due to the increased aggressiveness of the cells after two passages in the NSG. Summary of hCD45⁺ blasts in BM shown. Statistics calculated by unpaired t test. FIG. 8B. NSG mice were transplanted with TP53-mutated AML-012 and treated with Vehicle, CrEL-EM (0.1 mg/kg) or Lipo-EM (0.35 mg/kg) as in (FIG. 8A) and Kaplan-Meier survival curves of the mice are shown. FIG. 8C. NSG mice were transplanted with TP53-mutated AML-012 and treated with Vehicle or Lipo-EM (0.35 mg/kg) once every three days for 3 doses, then sacrificed for qPCR analysis of mRNA isolated from the sorted BM cells pooled from n=3 mice per group. Statistics are by t test (* p<0.05; ** p<0.01; *** p<0.001; **** p<0.0001; ns, not significant). Data are representative of two independent experiments. FIGS. 8D-E. NSG mice were transplanted with TP53-mutated AML-277 or TP53-mutated AML-172 and treated with Vehicle or Lipo-EM (0.1 mg/kg) once every other day for 10 doses. Kaplan-Meier survival curves of the mice xenografted with AML-277 (FIG. 8D) or AML-172 (FIG. 8E) are shown. FIGS. 8F-G. NSG mice were transplanted with TP53-mutated AML-277 or TP53-mutated AML-172 and treated with Vehicle or Lipo-EM (0.1 mg/kg) once every other day for 3 doses. Then, the mice were euthanized and qPCR of HIF1α targets was performed on the mRNA isolated from the sorted BM cells pooled from n=3 mice per group for either AML-277 (FIG. 8F) or AML-172 (FIG. 8G). Statistics are by t test (* p<0.05; ** p<0.01; *** p<0.001; **** p<0.0001; ns, not significant). Data are representative of two independent experiments.

### DETAILED DESCRIPTION

TP53 mutations have emerged as one of the most common driver mutations in human cancer and thus should, in theory, be one of the most attractive targets for cancer therapy. However, due to the broad scope of inactivation mutations, restoration of p53 function has not been successful in the clinic. As an alternative approach, it is of great interest to identify druggable pathways that are selectively activated after TP53 mutation.

The inventors have found that HIF-1α target genes are enriched in TP53-mutated, vs TP53-wild type, AML and that a HIF inhibitor, echinomycin, is broadly effective against multiple TP53-mutated AML samples in xenograft mouse models. In addition echinomycin was found to be more effective than Daunorubicin+Cytarabine (DNR+Ara-C) therapy. Importantly, while DNR+Ara-C therapy enriched for AML stem cells, echinomycin largely eliminated this population both in vitro and in vivo.

A critical barrier to the clinical development of echinomycin as an anti-cancer agent was the lack of a sensitive method to measure trace amounts of echinomycin in blood and in tissues for pharmacokinetic studies for dose optimization. Here, the inventors developed a sensitive and specific LC-MS/MS assay to measure echinomycin in plasma that is capable of detecting 0.025 ng/ml in mouse plasma or tissue extracts.

Furthermore, the invention describes a new echinomycin formulation with longer half-life and significantly improved therapeutic effect for TP53-mutated AML.

The inventors have made the surprising discovery that that HIF-1α pathway is activated in TP53-mutated AML, as its targets are coordinately increased. More importantly, they have shown that the HIF inhibitor, echinomycin, which inhibits HIF-1α activity by binding to the promoter region of its target genes, not only kills TP53-mutated AML blasts, but is even more active against the AML stem cell population. These data provide compelling evidence that HIF inhibitors may offer a new approach for unmet medical needs of patients with TP53-mutated leukemia.

The HIF inhibitor may be echinomycin, 2-methoxyestradiol, or geldanamycin. Echinomycin is a member of the quinoxaline family originally isolated from Streptomyces echinatus in 1957 and arguably the most potent HIF-1α inhibitor, with picomolar IC 50 in vitro. Echinomycin was never tested in human hematological malignancies until the inventors identified its function in the treatment of human AML, targeting cancer stem cells. Although echinomycin was used in several Phase II trials at a dose of 1200 μg/m 2 in humans, no pharmacokinetic (PK) data emerged since no method was available to measure drug concentration. A critical barrier to drug development was the lack of a sensitive method to measure trace amounts of echinomycin in blood and in tissues for pharmacokinetic studies for dose optimization. Here, the inventors have developed a sensitive and specific LC-MS/MS assay to measure echinomycin in plasma that is capable of detecting 0.025 ng/ml in mouse plasma or tissue extracts. This should greatly improve future efforts to develop what is likely the most effective inhibitor of the HIF pathway for cancer therapy.

The inventors tested PEGylated liposomal formulations containing HSPC:cholesterol:DSPE-mPEG2000 (57:38:5, mol:mol) for total drug exposure and obtained a more than 7-fold increase in total exposure within 24 hours. Importantly, and surprisingly, analysis of safety in mice showed that liposomal echinomycin used herein has significantly less toxicity despite increased exposure. These data show that by releasing echinomycin over an extended period, liposomes reduced exposure of normal tissues to toxicity associated with high concentrations of echinomycin within a very short period after dosing. This will not only increase drug availability for treatment of hematological malignancies, but also increase safety.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

A “peptide” or “polypeptide” is a linked sequence of amino acids and may be natural, synthetic, or a modification or combination of natural and synthetic.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

### 2. Hypoxia-Inducible Factor Inhibitor

Provided herein is an inhibitor of Hypoxia-Inducible Factor protein (HIF). The HIF inhibitor may be echinomycin, 2-methoxyestradiol, or geldanamycin.

a. HIF

The HIF may be a functional hypoxia-inducible factor, which may comprise a constitutive b subset and an oxygen-regulated a subunit. The HIF may be over-expressed in a broad range of human cancer types, which may be a breast, prostate, lung, bladder, pancreatic or ovarian cancer. While not being bound by theory, the increased HIF expression may be a direct consequence of hypoxia within a tumor mass. Both genetic and environmental factors may lead to the increased HIF expression even under the normoxia condition. Germline mutation of the von Hippel-Lindau gene (VHL), which may be the tumor suppressor for renal cancer, may prevent degradation HIF under normoxia. It may be possible to maintain constitutively HIF activity under normoxia by either upregulation of HIF and/or down regulation of VHL. The HIF may be HIF1α or HIF2α.

b. Echinomycin and Analogues

Echinomycin (NSC526417) is a member of the quinoxaline family originally isolated from Streptomyces echinatus. Echinomycin is a small-molecule that inhibits the DNA-binding activity of HIF-1α. The echinomycin may be a peptide antibiotic such as N,N′-(2,4,12,15,17,25-hexamethyl-11,24-bis(1-methylethyl)-27-(methylthio)-3,6,10,13,16,19,23,26-octaoxo-9,22-dioxa-28-thia-2,5,12,15,18,25-hexaazabicyclo(12.12.3)nonacosane-7,20-diyl)bis(2-quinoxalinecarboxamide). The echinomycin may be a microbially-derived quinoxaline antibiotic, which may be produced by Streptomyces echinatus. The echinomycin may have the following structure.

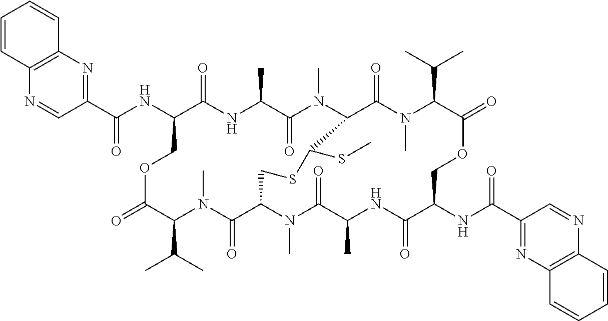 [SMILES: CSC1SC[C@H]2C(=O)N(C)[C@@H](C(C)C)C(=O)OC[C@@H](NC(=O)c3cnc4ccccc4n3)C(=O)N[C@@H](C)C(=O)N(C)[C@@H]1C(=O)N(C)[C@@H](C(C)C)C(=O)OC[C@@H](NC(=O)c1cnc3ccccc3n1)C(=O)N[C@@H](C)C(=O)N2C]

The echinomycin may have a structure as disclosed in U.S. Pat. No. 5,643,871, the contents of which are incorporated herein by reference. The echinomycin may also be an echinomycin derivative, which may comprise a modification as described in Gauvreau et al., Can J Microbiol, 1984; 30(6):730-8; Baily et al., Anticancer Drug Des 1999; 14(3):291-303; or Park and Kim, Bioorganic & Medicinal Chemistry Letters, 1998; 8(7):731-4, the contents of which are incorporated by reference. The echinomycin may also be a bis-quinoxaline analog of echinomycin.

Echinomycin analogues include compounds which due to their structural and functional similarity to echinomycin, exhibit effects on reduction of HIF-1α or HIF-2α activity, similar to that of echinomycin. Exemplary echinomycin analogues include YK2000 and YK2005 (Kim, J. B. et al., Int. J. Antimicrob. Agents, 2004 December; 24(6):613-615); Quinomycin G (Zhen X. et al., March Drugs, 2015 Nov. 18; 13(11):6947-61); 2QN (Bailly, C. et al., Anticancer Drug. Des., 1999 June; 14(3):291-303); and quinazomycin (Khan, A. W. et al., Indian J. Biochem., 1969 December; 6(4):220-1).

c. Microemulsion Echinomycin Drug Delivery Systems

Echinomycin is soluble in ethanol, alkalis, ketones, acetic acid and chloroform. It is insoluble in water. Echinomycin is therefore lipophilic, and generally readily associates with lipids, e.g., many of those used in the microemulsion drug-delivery systems of the present invention. In certain embodiments, echinomycin can also be formulated as a metal chelate.

The present application provides a microemulsion echinomycin drug delivery system for the treatment of proliferative disorders, such as TP53-mutated Acute Myeloid Leukemia, in which HIF-1α or HIF-2α is elevated. An emulsion is a mixture of two or more liquids that are normally immiscible (unmixable or unblendable). The microemulsion echinomycin drug delivery system may comprise liposomes, micelles or a mixture of liposomes and micelles. A liposome is a spherical vesicle with an aqueous solution core surrounded by a hydrophobic membrane, in the form of a lipid bilayer. Liposomes are most often composed of phospholipids, especially phosphatidylcholine, but may also include other lipids, so long as they are compatible with lipid bilayer structure. A typical micelle is a spherical vesicle formed by a single layer of amphiphilic molecules with the hydrophilic “head” regions of the amphiphilic molecules in contact with surrounding solvent, sequestering the hydrophobic single-tail regions in the center of the micelle.

The present application provides liposomal compositions encapsulating echinomycin, an echinomycin derivative, or an echinomycin analogue, and methods of using such compositions for the treatment of proliferative disorders. The echinomycin, echinomycin derivative, or echinomycin analogue formulations are preferably administered to a patient using a microemulsion drug-delivery system. Unless otherwise noted, the phrase “echinomycin formulation” should be interpreted to include microemulsion formulations containing echinomycin, an echinomycin derivative, or an echinomycin analogue.

In preferred embodiments, the microemulsion drug-delivery system used is a liposomal drug delivery system. In other embodiments, the microemulsion drug-delivery system used are composed of microparticles (or microspheres), nanoparticles (or nanospheres), nanocapsules, block copolymer micelles, or other polymeric drug delivery systems. In further embodiments, the drug delivery system used is a polymer-based, non-microemulsion drug delivery system such as hydrogels, films or other types of polymeric drug delivery system. In yet further embodiments, the echinomycin or echinomycin analogues are parenterally administered in a lipid-based solvent.

Microemulsion drug delivery vehicles, including liposomes, can be used to deliver echinomycin, echinomycin derivative, or echinomycin analogue into cells or patients with proliferative disorders. Echinomycin, echinomycin derivatives, or echinomycin analogues can be encapsulated (or incorporated) in any suitable microemulsion drug delivery vehicle that is capable of delivering the drug to target cells in vitro or in vivo.

As used herein, a microemulsion drug delivery vehicle is one that comprises particles that are capable of being suspended in a pharmaceutically acceptable liquid medium wherein the size range of the particles ranges from several nanometers to several micrometers in diameter. The microemulsion drug delivery systems contemplated by in the present application include those that substantially retain their microemulsion nature when administered in vivo. Microemulsion drug delivery systems include, but are not limited to, lipid-based and polymer-based particles. Examples of microemulsion drug delivery systems include liposomes, nanoparticles, (or nanospheres), nanocapsules, microparticles (or microspheres), and block copolymer micelles.

Liposomes bear many resemblances to cellular membranes and are contemplated for use in connection with the present invention as carriers for echinomycin and echinomycin analogues. They are widely suitable as both water- and lipid-soluble substances can be encapsulated, i.e., in the aqueous spaces and within the bilayer itself, respectively. The liposomal formulation of the liposome can be modified by those of skill in the art to maximize the solubility of echinomycin or any of its analogues based on their hydrophobicity.

Liposomes suitable for delivery of echinomycin, echinomycin derivatives, or echinomycin analogues include those composed primarily of vesicle-forming lipids. Appropriate vesicle-forming lipids for use in the present invention include those lipids which can form spontaneously into bilayer vesicles in water, as exemplified by the phospholipids.

Selection of the appropriate lipids for liposomes is governed by the factors of: (1) liposome stability, (2) phase transition temperature, (3) charge, (4) non-toxicity to mammalian systems, (5) encapsulation efficiency, (6) lipid mixture characteristics. It is expected that one of skill in the art who has the benefit of this disclosure could formulate liposomes according to the present invention which would optimize these factors. The vesicle-forming lipids of this type are preferably ones having two hydrocarbon chains, typically acyl chains, and a head group, either polar or nonpolar. The hydrocarbon chains may be saturated or have varying degrees of unsaturation. There are a variety of synthetic vesicle-forming lipids and naturally-occurring vesicle-forming lipids, including phospholipids, phosphoglycerides, glycolipids, such as the cerebrosides and gangliosides, sphingolipids, ether lipids, sterols, and caged phospholipids.

The liposome includes a liposomal shell composed of one or more concentric lipid monolayers or lipid bilayers. Thus, the lipid shell can be formed from a single lipid bilayer (i.e., the shell may be unilamellar) or several concentric lipid bilayers (i.e., the shell may be multilamellar). The lipids can be synthetic, semi-synthetic or naturally-occurring lipids, including phospholipids, tocopherols, steroids, fatty acids, glycoproteins such as albumin, anionic lipids and cationic lipids. The lipids may have an anionic, cationic or zwitterionic hydrophilic head group, and may be anionic, cationic lipids or neutral at physiologic pH.

Liposomal formulations may include a mixture of lipids. The mixture may comprise (a) a mixture of neutral and/or zwitterionic lipids; (b) a mixture of anionic lipids; (c) a mixture of cationic lipids; (d) a mixture of anionic lipids and cationic lipids; (e) a mixture of neutral or zwitterionic lipids and at least one anionic lipid; (f) a mixture of neutral or zwitterionic lipids and at least one cationic lipid; or (g) a mixture of neutral or zwitterionic lipids, anionic lipids, and cationic lipids. Further, the mixture may comprise saturated lipids, unsaturated lipids or a combination thereof. If an unsaturated lipid has two tails, both tails can be unsaturated, or it can have one saturated tail and one unsaturated tail.

In one embodiment, the lipid formulation is substantially free of anionic lipids, substantially free of cationic lipids, or both. In another embodiment, the lipid formulation is free of anionic lipids or cationic lipids or both. In one embodiment, the lipid formulation comprises only neutral lipids. Typically, a neutral lipid component is a lipid having two acyl groups (i.e., diacylphosphatidylcholine and diacylphosphatidylethanolamine). Lipids having a variety of acyl chain groups of varying chain length and degree of saturation are commercially available or may be isolated or synthesized by well-known techniques.

Exemplary neutral or zwitterionic phospholipids include, but are not limited to egg phosphatidylcholine (EPC), egg phosphatidylglycerol (EPG), egg phosphatidylinositol (EPI), egg phosphatidylserine (EPS), phosphatidylethanolamine (EPE), egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), 1-palmitoyl-2-myristoyl phosphatidylcholine (PMPC), 1-myristoyl-2-palmitoyl phosphatidylcholine (MPPC), dioleoylphosphatidylcholine (DOPC), dimyristoylphosphatidylcholine (DMPC), distearoylphosphatidylcholine (DSPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), dilauryloylphosphatidylcholine (DLPC), palmitoylstearoylphosphatidylcholine (PSPC), lysophosphatidylcholine (LPC), dilinoleoylphosphatidylcholine (DLPC), distearoylphophatidylethanolamine (DSPE), dimyristoylphosphatidylethanolamine (DMPE), dipalmitoyl phosphatidylethanolamine (DPPE), dioleylphosphatidylethanolamine (DOPE), dioleoyl phosphatidylethanolamine (DOPE), palmitoyloleoyl phosphatidylethanolamine (POPE), and palmitoylstearoylphosphatidylglycerol (PSPG), sterols, such as cholesterol and ergosterol; cholesterol esters, ceramides, cerebrosides, diacylglycerol, sphingosine, sphingomyelins, such as brain sphingomyelin, egg sphingomyelin, dipalmitoyl sphingomyelin, and distearoyl sphingomyelin dihydrosphingomyelin; and single acylated phospholipids, such as like mono-oleoyl-phosphatidylethanol amine (MOPE).

Zwitterionic lipids include, but are not limited to, acyl zwitterionic lipids and ether zwitterionic lipids. Examples of useful zwitterionic lipids are 1,2-dipalmitoyl-sn-glycero-3-phosphocholine (DPPC), 1,2-distearoyl-sn-glycero-3-phosphocholine (DSPC), 1,2-distearoyl-sn-glycero-3-phosphoethanolamine (DSPE), 1,2-dioleoyl-sn-glycero-3-phosphocholine (DOPC), 1,2-dioleoyl-sn-glycero-3-phosphatidylethanolamine (DOPE), 1,2-diphytanoyl-sn-glycero-3-phosphoethanolamine (DPyPE) and dodecylphosphocholine.

Exemplary anionic lipids include dihexadecylphosphate (DhP), phosphatidylinositols, phosphatidylserines, including diacylphosphatidylserines, such as dimyristoylphosphatidylserine, dipalmitoylphosphatidylserine; phosphatidylglycerols, such as dimyristoyl phosphatidylglycerol (DMPG), dipalmitoylphosphatidylglycerol (DPPG), distearoyl phosphatidylglycerol (DSPG), dioleoylphosphatidyl glycerol (DOPG), dilauryloylphosphatidyl glycerol (DLPG), distearyloylphosphatidyl glycerol (DSPG), and lysylphosphatidylglycerol (LPG); phosphatidylethanolamines, such as N-dodecanoyl phosphatidyl ethanoloamine, N-succinyl phosphatidylethanolamine, N-glutaryl phosphatidylethanolamine; phosphatidic acids, including diphosphatidyl glycerol and diacylphosphatidic acids, such as dimyristoyl phosphatic acid and dipalmitoyl phosphatic acid; cardiolipin, and cholesterol hemisuccinate (CHEMS).

Cationic lipids typically have a lipophilic moiety, such as a sterol, an acyl or diacyl chain, and where the lipid has an overall net positive charge. Preferably, the head group of the lipid carries the positive charge. Exemplary cationic lipids include N-[1-(2,3-dioleoyloxy)propyl]-N,N,N-trimethyl ammonium salts, also referred to as TAP lipids, for example as a methylsulfate salt. Suitable TAP lipids include, but are not limited to, DOTAP (dioleoyl-), DMTAP (dimyristoyl-), DPTAP (dipalmitoyl-), and DSTAP (distearoyl-). Other suitable cationic lipids include dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DO SPA), .β-alanyl cholesterol, cetyltrimethylammonium bromide (CTAB), diC14-amidine, N-tert-butyl-N′-tetradecyl-3-tetradecylaminopropionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide, 1-[2-(acyloxy)ethyl]2-alkyl(alkenyl)-3-(2-hydroxyethyl)-imidazolinium chloride derivatives, such as 1-[2-(9(Z)-octadecenoyloxy)ethyl]-2-(8(Z)-heptadecenyl-3-(2-hydroxyethyl)-imidazolinium chloride (DOTIM) and 1-[2-(hexadecanoyloxy)ethyl]-2-pentadecyl-3-(2-hydroxyethyl)imidazolinium chloride (DPTIM), and 2,3-dialkyloxypropyl quaternary ammonium derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIC-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DMRIE), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA).

Typically, a liposomal formulation according to the present application includes at least one lipid within the liposome that is pegylated, i.e., the lipid includes a polyethylene glycol moiety. Liposomes including PEGylated lipids will have PEG oriented so that it is present on at least the exterior of the liposome (but some PEG may also be exposed to the liposome's interior i.e. to the aqueous core). This orientation can be achieved by attaching the PEG to an appropriate part of the lipid. For example, in an amphiphilic lipid the PEG would be attached to the hydrophilic head, as it is this head which orients itself to the lipid bilayer's aqueous-facing exterior. PEGylation in this way can be achieved by covalent attachment of a PEG to a lipid using techniques known in the art.

Exemplary pegylated lipids include, but are not limited to distearoylphosphatidylethanolamine-polyethylene glycol (DSPE-PEG), including DSPE PEG (1000 MW), DSPE PEG (2000 MW) and DSPE PEG (5000 MW); dimyristoyl phosphatidylethanolamine-polyethylene glycol (DMPE-PEG), including DMPE PEG (1000 MW), DMPE PEG (2000 MW) and DMPE PEG (5000 MW); dipalmitoylglycerosuccinate polyethylene glycol (DPGS-PEG), including DPGS-PEG (1000 MW), DPGS (2000 MW) and DPGS (5000 MW); stearyl-polyethylene glycol, cholesteryl-polyethylene glycol, and ceramide-based pegylated lipids such as, N-octanoyl-sphingosine-1-{succinyl[methoxy(polyethylene glycol)MW]}, designated C8 PEG (MW) ceramide, where MW is 750, 2000, or 5000, or N-palmitoyl-sphingosine-1-{succinyl[methoxy(polyethylene glycol)MW]} or designated C16 PEG (MW) ceramide, where MW is 750, 2000, or 5000. Additional pegylated lipids can be obtained from Avanti Polar Lipids, Inc. (Alabaster, AL).

A liposome of the invention will typically include a large number of PEG moieties, which may be the same or different. The average molecular mass of the PEG in a liposome of the invention is above 350 Da but less than 5 kDa e.g., between 0.35-5 kDa, between 1-3 kDa, between 1-2-6 kDa, between 2-3 kDa, or 4-5 kDa, or preferably 2 kDa (PEG2000). The PEG will usually comprise linear polymer chains but, in some embodiments, the PEG may comprise branched polymer chains.

In some embodiments the PEG may be a substituted PEG e.g., in which one or more carbon atoms in the polymer is substituted by one or more alkyl, alkoxy, acyl or aryl groups. In other embodiments the PEG may include copolymer groups e.g., one or more propylene monomers, to form a PEG polypropylene polymer.

In certain embodiments, the liposome is formed from a mixture of one or more pegylated phospholipids and one or more additional neutral lipids. The molar percentage of the pegylated lipids may be between 0.1-20%. In some embodiments, the molar percentage of the pegylated lipids is between 1-9%, between 2-8%, and preferably between 5-6% of the total lipids in the composition.

As used herein, the “molar percentage” of lipid A in a mixture containing lipids A, B and C is defined as:

$$\frac{molar amount of A}{molar amount of A + molar amount of B + molar amount of C} \times 100 \%$$

In another embodiment, the liposome is formed from a lipid mixture comprising a pegylated phospholipid, a neutral phosphoglyceride, such as a phosphatidylcholine, phosphatidylserine, phosphatidylethanolamine, phosphatidylglycerol, or phosphatidylinositol; and a neutral sterol, such as cholesterol or ergosterol. In this embodiment, the molar percentage of the pegylated phospholipid may range from 1 to 10% or 3 to 6% of the total lipids; the amount of the neutral phosphoglyceride (to total lipids) may range from 20-60% or 30-50% or 33-43%; and the molar ratio of the neutral sterol may range from 35-75% or 45-65% or 50-60%.

In a particular embodiment, the liposome is formed from a mixture of DSPE-PEG (2000) (DSPE-mPEG2000), HSPC, and cholesterol. The molar percentage of HSPC, cholesterol, and DSPE-PEG (2000) may be 50-60%, 30-40%, and 1-5% respectively. In particular, the molar percentage of DSPE-PEG (2000) may be about 5%, the molar percentage of HSPC may be about 57%, and the molar percentage of cholesterol may be about 38%. In another embodiment, the molar percentage of DSPE-PEG (2000) may be about 5.3%, the molar percentage of HSPC may be about 56.3%, and the molar percentage of cholesterol may be about 38.4%. The HIF inhibitor and liposome may be present in the formulation at a 3% drug/lipid molar ratio. The liposome formulation may increase the circulation time of the HIF inhibitor described herein, which may be echinomycin. At the same time, the formulation may also have reduced toxicity.

As an alternative to pegylation, a lipid may be modified by covalent attachment of a moiety different from PEG. For example, in some embodiments a lipid may include a polyphosphazene. In some embodiments a lipid may include a poly(vinyl pyrrolidone). In some embodiments a lipid may include a poly(acryl amide). In some embodiments a lipid may include a poly(2-methyl-2-oxazoline). In some embodiments a lipid may include a poly(2-ethyl-2-oxazoline). In some embodiments a lipid may include a phosphatidyl polyglycerol. In some embodiments a lipid may include a poly[N-(2-hydroxypropyl)methacrylamide]. In some embodiments a lipid may include a polyalkylene ether polymer, other than PEG.

The fate and disposition of intravenously injected liposomes depend on their physical properties, such as size, fluidity and surface charge. They may persist in tissues for hours or days, depending on their composition, and half lives in the blood range from minutes to several hours. Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core. MLVs typically have diameters of from 0.5 to 4 μm. Sonication of MLVs results in the formation of large unilamellar vesicles (LUVs) with diameters in the range of 50-500 nm or small unilamellar vesicles (SUVs) with diameters less than 50 nm, typically in the range of 200 to 500 Å, containing an aqueous solution in the core.

Larger liposomes, such as MLVs and LUVs, can be taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow and lymphoid organs.

Liposomes of the present application are preferably SUVs with a diameter in the range of 60-180 nm, 80-160 nm, or 90-120 nm. A liposome of the present application can be part of a liposomal formulation comprising a plurality of liposomes in which liposomes within the plurality can have a range of diameters. In some embodiments, a liposomal formulation comprises at least 80%, at least 90%, or at least 95% of the liposomes have an average diameter in the range of 60-180 nm, 80-160 nm, 90-120 nm. Also, the diameters within the plurality may have a polydispersity index <0.2, <0.1 or <0.05. In some embodiments, the average diameter of the liposomes are determined using the Malvern Zetasizer method.

One way of increasing the circulation time of liposomes is by using liposomes derivatized with a hydrophilic polymer chain or polyalkylether, such as polyethyleneglycol (PEG)(See e.g., U.S. Pat. Nos. 5,013,556, 5,213,804, 5,225,212 and 5,395,619). The polymer coating reduces the rate of uptake of liposomes by macrophages and thereby prolongs the presence of the liposomes in the blood stream. This can also be used as a mechanism of prolonged release for the drugs carried by the liposomes. Accordingly, liposomal echinomycin formulations according to the present application preferably include one or more pegylated lipids.

One of skill in the art can select vesicle-forming lipid(s) that achieve a specified degree of fluidity or rigidity. The fluidity or rigidity of the liposome can be used to control factors such as the stability of the liposome in serum or the rate of release of the entrapped agent in the liposome. Liposomes having a more rigid lipid bilayer, or a liquid crystalline bilayer, are achieved by incorporation of a relatively rigid lipid. The rigidity of the lipid bilayer correlates with the phase transition temperature of the lipids present in the bilayer. Phase transition temperature is the temperature at which the lipid changes physical state and shifts from an ordered gel phase to a disordered liquid crystalline phase. Several factors affect the phase transition temperature of a lipid including hydrocarbon chain length and degree of unsaturation, charge and headgroup species of the lipid. A lipid having a relatively high phase transition temperature will produce a more rigid bilayer. Other lipid components, such as cholesterol, are also known to contribute to membrane rigidity in lipid bilayer structures. Cholesterol may be used to manipulate the fluidity, elasticity and permeability of the lipid bilayer. It is thought to function by filling in gaps in the lipid bilayer. In contrast, lipid fluidity is achieved by incorporation of a relatively fluid lipid, typically one having a lower phase transition temperature. Phase transition temperatures of many lipids are tabulated in a variety of sources, such as Avanti Polar Lipids catalogue and Lipidat by Martin Caffrey, CRC Press.

Phospholipids can form a variety of structures other than liposomes when dispersed in water, depending on the molar ratio of lipid to water. At low ratios the liposome is the preferred structure. The physical characteristics of liposomes depend on the pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes of the present application may be prepared to have substantially homogeneous sizes in a selected size range. One effective sizing method for REVs and MLVs involves extruding an aqueous suspension of the liposomes through a series of polycarbonate membranes having a selected uniform pore size in the range of 0.03 to 0.2 microns, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in Specialized Drug Delivery Systems-Manufacturing and Production Technology, (P. Tyle, Ed.) Marcel Dekker, New York, pp. 267-316 (1990)). Homogenization relies on shearing energy to fragment large liposomes into smaller ones. Other appropriate methods of down-sizing liposomes include reducing liposome size by vigorous agitation of the liposomes in the presence of an appropriate solubilizing detergent, such as deoxycholate.

Liposomes that have been sized to a range of about 0.2-0.4 microns may be sterilized by filtering the liposomes through a conventional sterilization filter, which is typically a 0.22 micron filter, on a high throughput basis. Other appropriate methods of sterilization will be apparent to those of skill in the art.

Non-toxicity of the lipids is also a significant consideration in the present application. Lipids approved for use in clinical applications are well-known to those of skill in the art. In certain embodiments, synthetic lipids, for example, may be preferred over lipids derived from biological sources due to a decreased risk of viral or protein contamination from the source organism.

The original method of forming liposomes involved first suspending phospholipids in an organic solvent and then evaporating to dryness until a dry lipid cake or film is formed. An appropriate amount of aqueous medium is added and the lipids spontaneously form multilamellar concentric bilayer vesicles (also termed multilamellar vesicles (MLVs). These MLVs can then be dispersed and reduced in size by mechanical means.

In spite of the water-insoluble nature of echinomycin, the inventors of the present application have found that stable liposomes can be formed by combining echinomycin and lipids in a polar solvent, such as ethanol, drying these components to form a film and then dispersing the liposomes in an aqueous medium. Thus, in one embodiment, after echinomycin and the lipids are thoroughly mixed in the organic solvent, the solvent is removed using e.g., a rotary evaporator, thereby resulting in a dried lipid film. The dried lipid film is hydrated and solubilized in a suitable buffer (e.g., PBS, pH 7.4), thereby resulting in a lipid suspension. The lipid suspension is then repetitively extruded through polycarbonate filters using an Avanti Mini-Extruder to achieve a desired size range of liposomes. The liposomes are then sterilized by filtration (0.45- or 0.2-μm sterile filters). Water-soluble echinomycin analogues can be passively entrapped by hydrating a lipid film with an aqueous solution containing the water-soluble echinomycin analogue.

Echinomycin may be localized within the lipid bilayer, between the two leaflets of the lipid bilayer, within the internal core space, upon either face of the bilayer, within or upon the PEG moiety of the liposome, or a combination thereof. An alternate method for creating large unilamellar vesicles (LUVs) is the reverse-phase evaporation process, described, for example, in U.S. Pat. No. 4,235,871. This process generates reverse-phase evaporation vesicles (REVs), which are mostly unilamellar but also typically contain some oligolamellar vesicles. In this procedure a mixture of polar lipid in an organic solvent is mixed with a suitable aqueous medium. A homogeneous water-in-oil type of emulsion is formed and the organic solvent is evaporated until a gel is formed. The gel is then converted to a suspension by dispersing the gel-like mixture in an aqueous media.

In in alternate embodiment, echinomycin, echinomycin derivatives, or echinomycin analogues may be conjugated to the surface of the liposomal bilayer. In one embodiment, echinomycin is covalently attached to a liposome by amide conjugation. For example, phospholipids with hydroxyl functional groups can be conjugated to one of the amine groups present in echinomycin or one of its analogues.

Liposomal formulation according to the present invention will have sufficient long-term stability to achieve a shelf-life of at least 3 months, at least 6 months, at least 12 months, at least 24 months or at least 48 months at room temperature or refrigeration temperature (e.g., 4° C.).

In some alternative embodiments, the echinomycin or echinomycin analogue may be encapsulated in a protective wall material that is polymeric in nature rather than lipid-based. The polymer used to encapsulate the bioactive agent is typically a single copolymer or homopolymer. The polymeric drug delivery system may be microemulsion or non-microemulsion in nature.

Microemulsion polymeric encapsulation structures include microparticles, microcapsules, microspheres, nanoparticles, nanocapsules, nanospheres, block copolymer micelles, and the like. Both synthetic polymers, which are made by man, and biopolymers, including proteins and polysaccharides, can be used in the present invention. The polymeric drug delivery system may be composed of biodegradable or non-biodegradable polymeric materials, or any combination thereof.

As used herein, a “microemulsion” refers to an emulsion comprising microspheres that are of regular or semi-regular shape with a diameter of from about 10 nm to 500 μm. In some embodiments, the microemulsion of the present application contains liposomes with diameters in the range of 20-400 nm, 30-300 nm, 50-200 nm, 60-150 nm or 80-120 nm.

In some embodiments, the microemulsion of the present application comprises micelles having a shell composed of a single layer of amphiphilic molecules. The inner core of the micelle creates a hydrophobic microenvironment for non-polar drugs, while the hydrophilic shell provides a stabilizing interface between the micelle core and the aqueous medium. The properties of the hydrophilic shell can be adjusted to both maximize biocompatibility and avoid reticuloendothelial system uptake and renal filtration. The size of the micelles is usually between 10 nm and 100 nm.

Non-microemulsion polymeric drug-delivery systems including films, hydrogels and “depot” type drug delivery systems are also contemplated by the present invention. Such non-microemulsion polymeric systems can also be used in the present invention in conjunction with parenteral injection, particularly where the non-microemulsion drug delivery system is placed in proximity to the targeted cancerous tissue. As used herein, a “hydrogel” means a solution of polymers, sometimes referred to as a sol, converted into gel state by small ions or polymers of the opposite charge or by chemical crosslinking. A “polymeric film” refers to a polymer-based film generally from about 0.5 to 5 mm in thickness which is sometimes used as a coating.

In certain embodiments the liposomes, microparticles, nanoparticles, microcapsules, block copolymer micelles or other polymeric drug delivery vehicles comprising echinomycin or an echinomycin analogue can be coated, conjugated to or modified with a cell-specific targeting ligand. By linking a delivery vehicle to a cell-targeting ligand, delivery of echinomycin can be directed to a target cell population which binds to the cell-targeting ligand or targeting ligand. As used herein, a “targeting ligand” includes any ligand which causes a liposome to associate with the target cell-type to an enhanced degree over non-targeted tissues

Targeting ligands, such as antibodies or antibody fragments can be used to bind to the liposome surface and to direct the antibody and its drug contents to specific antigenic receptors located on a particular cell-type surface (See e.g., Mastrobattista et al., 1999). Carbohydrate determinants (glycoprotein, lectin and glycolipid cell-surface components that play a role in cell-cell recognition, interaction and adhesion) can also be used as targeting ligands as they have potential in directing liposomes to particular cell types. Certain proteins can be used as targeting ligands, usually ones that are recognized by self-surface receptors of the targeted tissue. For example, a ligand that binds to a cell-surface receptor that is overexpressed in particular cancer cells might be used to increase uptake of liposomes by the target tissue. Cell surface receptors that are endocytosed will be preferred in certain embodiments. When combined with pegylated liposomes, the targeting ligand is often attached to the end of the hydrophilic polymer that is exposed to the aqueous medium. Alternately, liposomes can incorporate fusogenic proteins, e.g., fusogenic proteins derived from viruses, which induce fusion of the liposome with the cellular membrane.

In certain embodiments, the targeting ligand is a cell surface receptor that is endocytosed by the target cell. Appropriate targeting ligands for use in the present application include any ligand that causes increased binding or association of liposomes with cell-surface of the target cells over non-target cells. The targeting ligand can be a small molecule, peptide, ligand, antibody fragment, aptamer or synbody. A synbody is a synthetic antibody produced from a library comprised of strings of random peptides screened for binding to target proteins of interest and are described in U.S. 2011/0143953. An aptamer is a nucleic acid version of an antibody that comprises a class of oligonucleotides that can form specific three dimensional structures exhibiting high affinity binding to a wide variety of cell surface molecules, proteins, and/or macromolecular structures. Exemplary cell targeting ligands include, but are not limited to, small molecules (e.g., folate, adenosine, purine) and large molecules (e.g., peptide or antibody) that bind to (and target) e.g., epidermal dendritic cells as further described below.

Exemplary antibody or antibody derived fragments may include any member of the group consisting of: IgG, antibody variable region; isolated CDR region; single chain Fv molecule (scFv) comprising a VH and VL domain linked by a peptide linker allowing for association between the two domains to form an antigen binding site; bispecific scFv dimer; minibody comprising a scFv joined to a CH3 domain; diabody (dAb) fragment; single chain dAb fragment consisting of a VH or a VL domain; Fab fragment consisting of VL, VH, CL and CH1 domains; Fab′ fragment, which differs from a Fab fragment by the addition of a few residues at the carboxyl terminus of the heavy chain CH1 domain, including one or more cysteines from the antibody hinge region; Fab′-SH fragment, a Fab′ fragment in which the cysteine residue(s) of the constant domains bear a free thiol group; F(ab′)2, bivalent fragment comprising two linked Fab fragments; Fd fragment consisting of VH and CH1 domains; derivatives thereof; and any other antibody fragment(s) retaining antigen-binding function. Fv, scFv, or diabody molecules may be stabilized by the incorporation of disulphide bridges linking the VH and VL domains. When using antibody-derived fragments, any or all of the targeting domains therein and/or Fc regions may be “humanized” using methodologies well known to those of skill in the art. In some embodiments, the antibody may be modified to remove the Fc region.

d. Pharmaceutical Composition

Also provided is a pharmaceutical composition comprising the HIF inhibitor. Pharmaceutical compositions of the present invention comprising echinomycin, an echinomycin derivative, or an echinomycin analogue and a microemulsion drug delivery carrier such as a liposome are prepared according to standard techniques. They can further comprise a pharmaceutically acceptable carrier. As used herein, the term “pharmaceutically acceptable” refers to a molecular entity or composition that does not produce an adverse, allergic or other untoward reaction when administered to an animal or a human, as appropriate. The term “pharmaceutically acceptable carrier”, as used herein, includes any and all solvents, dispersion media, coatings, antibacterial and/or antifungal agents, isotonic and absorption delaying agents, buffers, excipients, binders, lubricants, gels, surfactants and the like, that may be used as a media for a pharmaceutically acceptable substance.

Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents.

These compositions can be sterilized by conventional sterilization techniques that are well-known to those of skill in the art. Sufficiently small liposomes, for example, can be sterilized using sterile filtration techniques.

Formulation characteristics that can be modified include, for example, the pH and the osmolality. For example, it may be desired to achieve a formulation that has a pH and osmolality similar to that of human blood or tissues to facilitate the formulation's effectiveness when administered parenterally. Alternatively, to promote the effectiveness of the disclosed compositions when administered via other administration routes, alternative characteristics may be modified.

Buffers are useful in the present invention for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present invention include sodium or potassium buffers, including sodium phosphate, potassium phosphate, sodium succinate and sodium citrate.

Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present invention include a pH of about 2.0 to a pH of about 12.0.

In some embodiments, it will also be advantageous to employ surfactants in the presently disclosed formulations, where those surfactants will not be disruptive of the drug-delivery system used. Surfactants or anti-adsorbents that prove useful include polyoxyethylenesorbitans, polyoxyethylenesorbitan monolaurate, polysorbate-20, such as Tween-20™, polysorbate-80, polysorbate-20, hydroxycellulose, genapol and BRIJ surfactants. By way of example, when any surfactant is employed in the present invention to produce a parenterally administrable composition, it is advantageous to use it in a concentration of about 0.01 to about 0.5 mg/ml.

Additional useful additives are readily determined by those of skill in the art, according to particular needs or intended uses of the compositions and formulator. One such particularly useful additional substance is sodium chloride, which is useful for adjusting the osmolality of the formulations to achieve the desired resulting osmolality. Particularly preferred osmolalities for parenteral administration of the disclosed compositions are in the range of about 270 to about 330 mOsm/kg. The optimal osmolality for parenterally administered compositions, particularly injectables, is approximately 3000 sm/kg and achievable by the use of sodium chloride in concentrations of about 6.5 to about 7.5 mg/ml with a sodium chloride concentration of about 7.0 mg/ml being particularly effective.

Echinomycin-containing liposomes or echinomycin-containing microemulsion drug-delivery vehicles can be stored as a lyophilized powder under aseptic conditions and combined with a sterile aqueous solution prior to administration. The aqueous solution used to resuspend the liposomes can contain pharmaceutically acceptable auxiliary substances as required to approximate physical conditions, such as pH adjusting and buffering agents, tonicity adjusting agents and the like, as discussed above.

In other embodiments the echinomycin-containing liposomes or echinomycin-containing microemulsion drug-delivery vehicle can be stored as a suspension, preferable an aqueous suspension, prior to administration. In certain embodiments, the solution used for storage of liposomes or microemulsion drug carrier suspensions will include lipid-protective agents which protect lipids against free-radical and lipid-peroxidative damage on storage. Suitable protective compounds include free-radical quenchers such as alpha-tocopherol and water-soluble iron-specific chelators, such as ferrioxamine.

The pharmaceutical composition may be in the form of tablets or lozenges formulated in a conventional manner. For example, tablets and capsules for oral administration may contain conventional excipients may be binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers may be lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants may be potato starch and sodium starch glycollate. Wetting agents may be sodium lauryl sulfate. Tablets may be coated according to methods well known in the art.

The pharmaceutical composition may also be liquid formulations such as aqueous or oily suspensions, solutions, emulsions, syrups, and elixirs. The pharmaceutical composition may also be formulated as a dry product for constitution with water or other suitable vehicle before use. Such liquid preparations may contain additives such as suspending agents, emulsifying agents, nonaqueous vehicles and preservatives. Suspending agents may be sorbitol syrup, methyl cellulose, glucose/sugar syrup, gelatin, hydroxyethyl cellulose, carboxymethyl cellulose, aluminum stearate gel, and hydrogenated edible fats. Emulsifying agents may be lecithin, sorbitan monooleate, and acacia. Nonaqueous vehicles may be edible oils, almond oil, fractionated coconut oil, oily esters, propylene glycol, and ethyl alcohol. Preservatives may be methyl or propyl p-hydroxybenzoate and sorbic acid.

The pharmaceutical composition may also be formulated as suppositories, which may contain suppository bases such as cocoa butter or glycerides. The pharmaceutical composition may also be formulated for inhalation, which may be in a form such as a solution, suspension, or emulsion that may be administered as a dry powder or in the form of an aerosol using a propellant, such as dichlorodifluoromethane or trichlorofluoromethane. Agents provided herein may also be formulated as transdermal formulations comprising aqueous or nonaqueous vehicles such as creams, ointments, lotions, pastes, medicated plaster, patch, or membrane.

The pharmaceutical composition may also be formulated for parenteral administration such as by injection, intratumor injection or continuous infusion. Formulations for injection may be in the form of suspensions, solutions, or emulsions in oily or aqueous vehicles, and may contain formulation agents including, but not limited to, suspending, stabilizing, and dispersing agents. The pharmaceutical composition may also be provided in a powder form for reconstitution with a suitable vehicle including, but not limited to, sterile, pyrogen-free water.

The pharmaceutical composition may also be formulated as a depot preparation, which may be administered by implantation or by intramuscular injection. The pharmaceutical composition may be formulated with suitable polymeric or hydrophobic materials (as an emulsion in an acceptable oil, for example), ion exchange resins, or as sparingly soluble derivatives (as a sparingly soluble salt, for example).

(1) Administration

Administration of the pharmaceutical composition may be orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. For veterinary use, the agent may be administered as a suitably acceptable formulation in accordance with normal veterinary practice. The veterinarian can readily determine the dosing regimen and route of administration that is most appropriate for a particular animal. The pharmaceutical composition may be administered to a human patient, cat, dog, large animal, or an avian.

In certain embodiments, the composition can be formulated as a depot preparation. Such long acting formulations may be administered by implantation at an appropriate site or by parenteral injection, particularly intratumoral injection or injection at a site adjacent to cancerous tissue.

When echinomycin, echinomycin derivative, or an echinomycin analog is encapsulated in a liposome or other microemulsion drug-delivery vehicle, any effective amount of the echinomycin or echinomycin may be administered. Preferably, the liposomal formulations or other microemulsion drug-delivery vehicles containing echinomycin, an echinomycin derivative, or an echinomycin analogue are administered by parenteral injection, including intravenous, intraarterial, intramuscular, subcutaneous, intra-tissue, intranasal, intradermal, instillation, intracerebral, intrarectal, intravaginal, intraperitoneal, intratumoral.

Intravenous administration of liposomal echinomycin has been tolerated by mice at doses of approximately 1 mg/kg of body weight and no LD₅₀ value has been reached. In contrast, free echinomycin has an LD₅₀ value of 0.629 mg/kg.

Liposomal preparations or other microemulsion delivery vehicles can be lyophilized and stored as sterile powders, preferably under vacuum, and then reconstituted in bacteriostatic water (containing, for example, benzyl alcohol preservative) or in sterile water prior to injection. Pharmaceutical compositions may be formulated for parenteral administration by injection e.g., by bolus injection or continuous infusion.

The delivery vehicle may be administered to the patient at one time or over a series of treatments and may be administered to the patient at any time from diagnosis onwards. The delivery vehicle may be administered as the sole treatment or in conjunction with other drugs or therapies useful in treating the condition in question.

The present invention is further illustrated by the following examples which should not be construed as limiting. The contents of all references, patents and published patent applications cited throughout this application, as well as the FIGS. and Tables are incorporated herein by reference. Those of skill in the art should, in light of the present disclosure, appreciate that many changes can be made in the specific embodiments which are disclosed and still obtain a like or similar result without departing from the spirit and scope of the invention.

The pharmaceutical composition may be administered simultaneously or metronomically with other treatments. The term “simultaneous” or “simultaneously” as used herein, means that the pharmaceutical composition and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The pharmaceutical composition may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, and 1 mins. The pharmaceutical composition may be administered at any point prior to a second treatment of the pharmaceutical composition including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

The pharmaceutical composition may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, and 120 hr. The pharmaceutical composition may be administered at any point prior after a pharmaceutical composition treatment of the agent including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

e. Dosage

The pharmaceutical composition may be administered in a therapeutically effective amount of the HIF inhibitor to a mammal in need thereof. The therapeutically effective amount required for use in therapy varies with the nature of the condition being treated, the length of time desired to inhibit HIF activity, and the age/condition of the patient.

Echinomycin/echinomycin derivative/echinomycin analogue dosages can be tested in a suitable animal model as further described below. As a general proposition, a therapeutically effective amount of echinomycin, echinomycin analogue or other anti-cancer agent will be administered in a range from about 10 ng/kg body weight/day to about 100 mg/kg body weight/day whether by one or more administrations. In a particular embodiment, each fusion protein or expression vector is administered in the range of from about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 11 μg/kg body weight/day to about 10 mg/kg body weight/day, about 11 μg/kg body weight/day to about 1 mg/kg body weight/day, about 11 μg/kg body weight/day to about 100 μg/kg body weight/day, about 11 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In some embodiments, echinomycin is administered at a body surface area (BSA)-based dose of 10-30,000 μg/m², 100-30,000 μg/m², 500-30,000 μg/m², 1000-30,000 μg/m², 1500-30,000 μg/m², 2000-30,000 μg/m², 2500-30,000 μg/m², 3000-30,000 μg/m², 3500-30,000 μg/m², 4000-30,000 μg/m², 100-20,000 μg/m², 500-20,000 μg/m², 1000-20,000 μg/m², 1500-20,000 μg/m², 2000-20,000 μg/m², 2500-20,000 μg/m², 3000-20,000 μg/m², 3500-20,000 μg/m², 100-10,000 μg/m², 500-10,000 μg/m², 1000-10,000 μg/m², 1500-10,000 μg/m², 2000-10,000 μg/m², or 2500-10,000 μg/m².

In other embodiments, echinomycin is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μs to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The fusion protein or expression vector may be administered daily, every 2, 3, 4, 5, 6 or 7 days, or every 1, 2, 3 or 4 weeks.

In other particular embodiments, the amount of echinomycin may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient.

The therapeutic agents in the pharmaceutical compositions may be formulated in a “therapeutically effective amount”. A “therapeutically effective amount” refers to an amount effective, at dosages and for periods of time necessary, to achieve the desired therapeutic result. A therapeutically effective amount of the liposomal formulation or other microemulsion drug-delivery vehicle may vary depending on the condition to be treated, the severity and course of the condition, the mode of administration, the bioavailability of the particular agent(s), the ability of the delivery vehicle to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the delivery vehicle is outweighed by the therapeutically beneficial effects.

The dose may be a non-toxic dose. The dose may also be one at which HIF activity is inhibited, but at which c-Myc activity is unaffected. In general, however, doses employed for adult human treatment typically may be in the range of 1-100 μg/m 2 per day, or at a threshold amount of 1-100 μg/m 2 per day or less, as measured by a body-surface adjusted dose. The desired dose may be conveniently administered in a single dose, or as multiple doses administered at appropriate intervals, for example as two, three, four or more sub-doses per day. Multiple doses may be desired, or required.

The dosage may be a dosage such as about 1 μg/m², 2 μg/m², 3 μg/m₂, 4 μg/m₂, 5 μg/m₂, 6 μg/m², 7 μg/m², 8 μg/m², 9 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 70 μg/m², 80 μg/m², 90 μg/m², 100 μg/m², 200 μg/m², 300 μg/m², 400 μg/m², 500 μg/m², 600 μg/m², 700 μg/m², 800 μg/m², 900 μg/m², 1000 μg/m², 1100 μg/m 2, or 1200 μg/m 2, and ranges thereof.

The dosage may also be a dosage less than or equal to about 1 μg/m², 2 μg/m², 3 μg/m², 4 μg/m², 5 μg/m², 6 μg/m², 7 μg/m², 8 μg/m², 9 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 70 μg/m², 80 μg/m², 90 μg/m², 100 μg/m², 200 μg/m², 300 μg/m², 400 μg/m², 500 μg/m², 600 μg/m², 700 μg/m², 800 μg/m², 900 μg/m², 1000 μg/m², 1100 μg/m², or 1200 μg/m², and ranges thereof

f. Coadministration

In certain embodiments, the HIF-1α inhibitors of the present application may be combined with standard cancer treatments (e.g., surgery, radiation, and chemotherapy). Such an approach is predicated on the fact that HIFs are known to mediate resistance to radiation therapy and chemotherapy (Semenza, Trends Pharmacol Sci. 2012 April; 33(4): 207-214). For example, evidence indicates that HIF-1 activity may contribute to the development of resistance to novel targeted therapies, such as imatinib treatment of chronic myeloid leukemia. Specifically, HIF-1 appears to mediate resistance to imatinib through metabolic reprogramming, by activating expression of transketolase and thereby increasing glucose flux through the non-oxidative arm of the pentose phosphate pathway. The switch from oxidative to reductive metabolism that is mediated by HIF-1 has the effect of reducing cellular ROS levels, which may increase resistance to cytotoxic chemotherapy (Semenza, 2012).

In certain embodiments, echinomycin, its derivatives or its analogues may be administered in synergistic combinations with one or more other chemotherapeutic or anti-cancer agents. In these instances, it may be possible to reduce the dose of the chemotherapeutic or anti-cancer agents administered. An example of such a combination is echinomycin in combination with imatinib for the treatment of leukemia. It is believed that the combined use of HIF-1α inhibition and chemotherapy can reverse the negative effects of resistance to radiation therapy, chemotherapy, and/or apoptosis, as well as angiogenesis, stem cell maintenance, metabolic reprogramming, autocrine growth factor signaling, epithelial-mesenchymal transition, invasion, and metastasis.

As used herein, the phrase “anti-cancer agent” refers to a “small molecule drug” or a protein or antibody that can reduce the rate of cancer cell growth or induce or mediate the death (e.g., necrosis or apoptosis) of cancer cells in a subject (e.g., a human). The phrase “small molecule drug” refers to a molecular entity, often organic or organometallic, that is not a polymer, that has medicinal activity, and that has a molecular weight less than about 2 kDa, less than about 1 kDa, less than about 900 Da, less than about 800 Da or less than about 700 Da. The term encompasses most medicinal compounds termed “drugs” other than protein or nucleic acids, although a small peptide or nucleic acid analog can be considered a small molecule drug. Examples include chemotherapeutic anticancer drugs and enzymatic inhibitors. Small molecules drugs can be derived synthetically, semi-synthetically (i.e., from naturally occurring precursors), or biologically.

The anti-cancer agent may be an alkylating agent; an anthracycline antibiotic; an anti-metabolite; a detoxifying agent; an interferon; a polyclonal or monoclonal antibody; an EGFR inhibitor; a HER2 inhibitor; a histone deacetylase inhibitor; a hormone or anti-hormonal agent; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue or combination thereof.

### 3. Method of Treating a Hematologic Cancer

Provided herein is a method of treating a hematologic cancer. The methods of the present invention are useful for the treatment of proliferative disorders in all mammalian subjects, particularly human patients. As used herein, a “patient” is a human patient. The method may comprise administering a HIF inhibitor to a mammal in need thereof. The mammal may be a human patient. The hematologic cancer may be lymphoma or leukemia. The hematologic cancer may be treated by inhibiting a maintenance or survival function of a CSC. Without being bound by theory inhibiting HIF may target both the cancer stem cell and cancer resistance.

The leukemia may be TP53-mutated acute myeloid leukemia (AML). Somatic TP53 mutations are frequently detected in a variety of cancers, with different frequencies dependent on the cancer type. TP53 mutations are found in acute myeloid leukemia (AML) patients with a frequency of over 10%, especially in cases with complex karyotypes, and are found at even higher frequencies in therapy-related AML (between 20-40%). Overall, TP53 mutations are associated with very poor prognosis, with poor responses to chemotherapy and allogeneic stem cell transplantation. Response rates to hypomethylating agents are higher, but responses are not durable. Restoration of p53 function is a possible strategy to suppress cancer growth, but no targeted therapy is available clinically to restore p53 function.

Further without being bound by theory, the CSC in the hematologic cancer may require self-renewal, which may be similar to the requirement in tissue cells. The CSC may require a hypoxic environment, and exposure to a high level of oxygen may reduce CSC function. Self-renewal of CSC function may be strongly inhibited by drugs targeting the HIF pathway. CSC may be addicted to the HIF, which may be associated with over-expression of HIF and down-regulation of VHL. HIF over-expression and VHL down-regulation may be critical in the maintenance of CSC.

### Example 1

### CD34⁺CD38⁻ Human Primary TP53-Mutated AML Cells are More Sensitive to Echinomycin

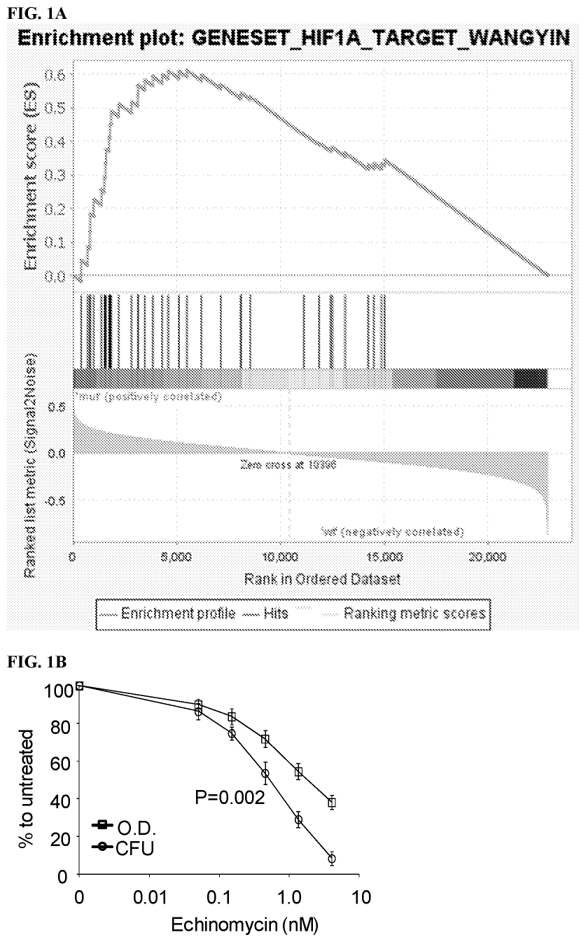

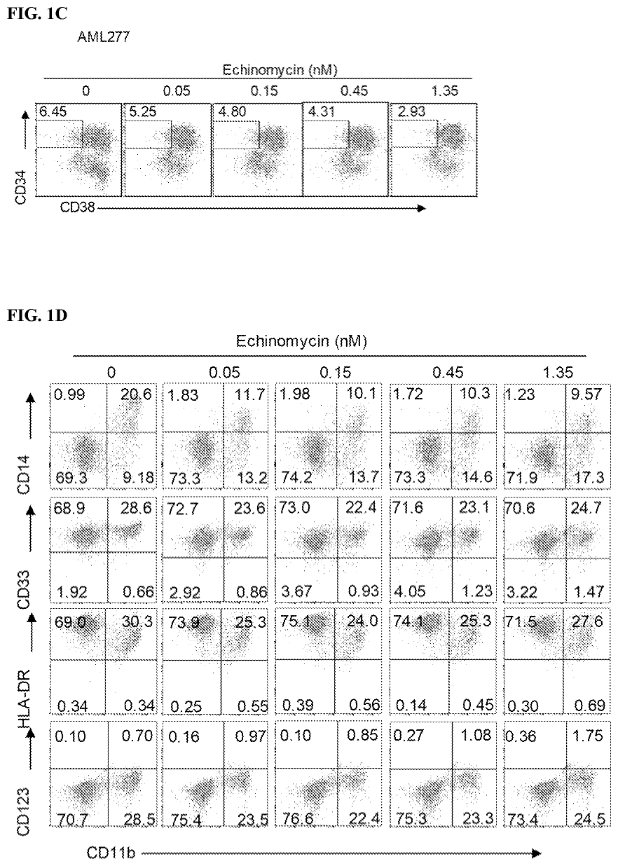

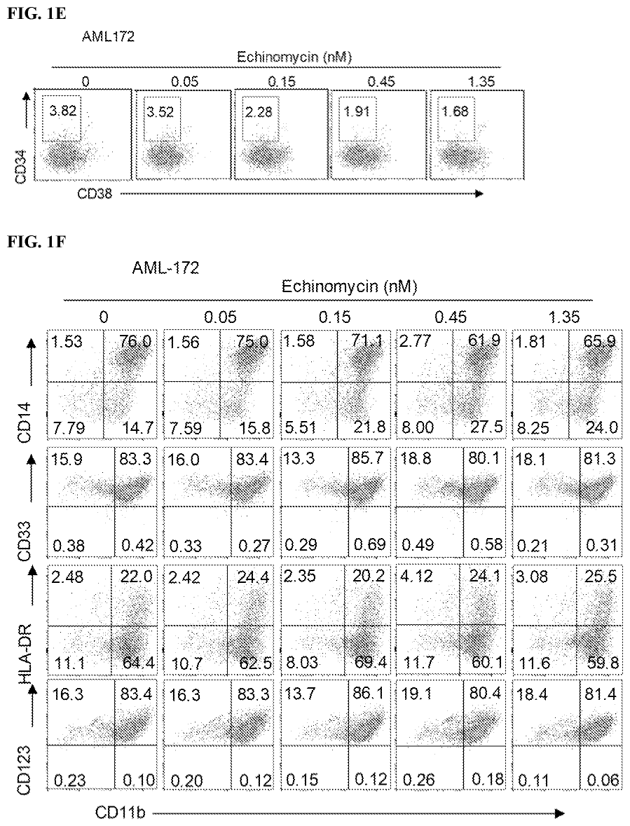

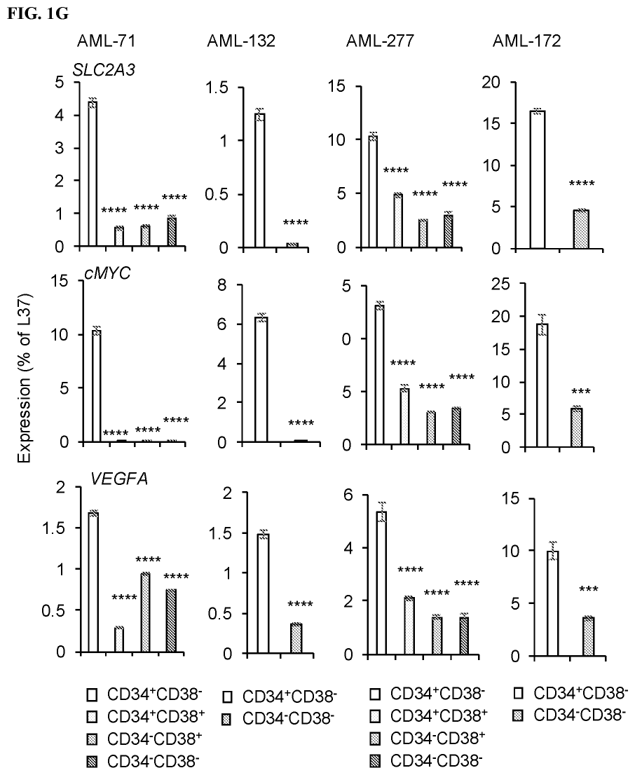

We analyzed a published dataset by GSEA for gene sets that are significantly enriched in 32 AML samples harboring TP53 mutant compared to 419 patients TP53 wild type patients with RNA-seq data. Among 50 hallmark gene sets from Molecular Signatures Database (MSigDB), and using FDR<0.1 as the threshold, we identified 8 sets that were enriched in patients with TP53-mutated AML samples. Among them, HIF-1α target genes were most significantly enriched (Table 1 and FIG. 1A).

TABLE 1

| GeneSets | NES | NOM p-val |
|---|---|---|
| HALLMARK_MITOTIC_SPINDLE | 2.08 | 0 |
| HALLMARK_G2M_CHECKPOINT | 2 | 0.015 |
| HIF1A_TARGET_GENES | 1.88 | 0.029 |
| HALLMARK_UV_RESPONSE_UP | 1.88 | 0.003 |
| HALLMARK_CHOLESTEROL_HOMEOSTASIS | 1.87 | 0.008 |
| HALLMARK_ESTROGEN_RESPONSE_LATE | 1.83 | 0 |
| HALLMARK_MTORC1_SIGNALING | 1.76 | 0.018 |
| HALLMARK_HEDGEHOG_SIGNALING | 1.75 | 0 |

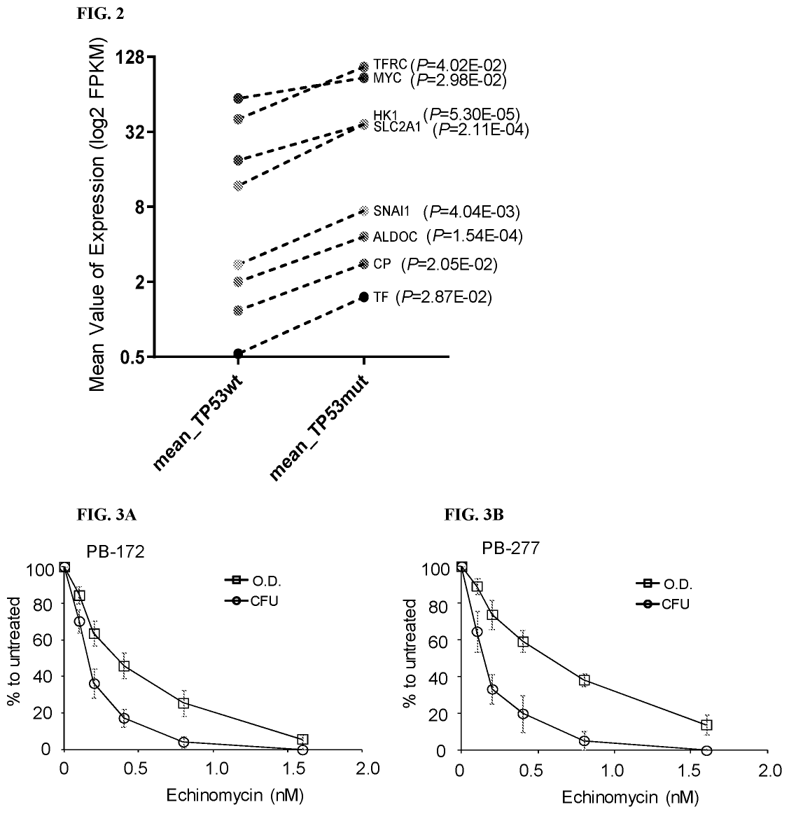

We also compared the expression of HIF-1α target genes in AML patients from The Cancer Genome Atlas (TCGA). Patients with TP53 mutation had enriched activity of HIF1A target genes compared to TP53 wild type patients, which was confirmed by the dramatic upregulation of eight H/F/A-target marker genes (FIG. 2). We tested the effect of echinomycin, an inhibitor of HIF-1α, on seven primary TP53-mutated AML samples by incubating the cells with 0.05 to 4.05 nM echinomycin and measuring cell viability and colony formation by MTT and colony-forming cell assays, respectively. Echinomycin had potent cytotoxic effects on TP53-mutated AML-277, with EC₅₀ of 2.075 nM after only 24 hrs incubation time (FIG. 1B), and EC₅₀ of about 0.5 nM if incubated for 48 hrs (FIG. 3). Compared to AML blasts, colony-forming unit (CFU) AML subsets were approximately 2.5-fold more sensitive to echinomycin, exhibiting EC₅₀s of about 0.883 nM (FIG. 1B). We tested six additional TP53-mutated AML samples and found similar results in sensitivity to echinomycin in MTT and CFU, summarized in Table 2. The IC₅₀s in the MTT assay are in the range of 0.656 nM to 3.021 nM, while EC₅₀s in the CFU assay are in the range of 0.113 to 0.833 nM (Table 2).

TABLE 2
IC₅₀ of MTT and CFU (Mean ± SD) of TP53-mutated and -wild type AML cells treated with Echinomycin

| | TP53-mutated AML | | | | | | | TP53-wild type AML | |
|---|---|---|---|---|---|---|---|---|---|
| | AML-12 | AML-83 | AML-135 | AML-147 | AML-172 | AML-253 | AML-277 | AML-71 | AML-132 |
| IC₅₀ (MTT, nM) | 3.021 ± | 0.656 ± | 2.017 ± | 1.252 ± | 2.405 ± | 1.274 ± | 2.075 ± | 3.438 ± | 3.276 ± |
| | 1.145 | 0.061 | 0.205 | 0.325 | 0.344 | 0.148 | 0.213 | 1.295 | 0.979 |
| IC₅₀ (CFU, nM) | 0.152 ± | 0.113 ± | 0.232 ± | 0.237 ± | 0.376 ± | 0.326 ± | 0.833 ± | 0.184 ± | 0.175 ± |
| | 0.021 | 0.067 | 0.061 | 0.014 | 0.121 | 0.078 | 0.039 | 0.179 | 0.098 |

These data demonstrated a critical role for HIF-1α in CFU activity in seven primary TP53-mutated AML samples. To test whether echinomycin selectively targeted subsets of the TP53-mutated AML cells in the CFU assay, we analyzed the percentage of CD34⁺CD38⁻ cells from the colonies on day 10 after treatment with echinomycin. CD34⁺CD38⁻ AML subsets, which are leukemia stem cells, were decreased in a concentration-dependent manner in primary TP53-mutated AML, and were decreased more than two-fold in the colonies treated with 1.35 nM of Echinomycin compared to vehicle-treated cells (FIG. 1C, E). However, there was no significant difference in the frequency of subsets with other leukemia markers (FIG. 1D, F). These data suggested that HIF-1α activity is required more for maintenance of the CD34⁺CD38⁻ AML subset compared to bulk leukemia cells. We previously reported that enrichment of HIF-1α in TP53 wild type CD34⁺CD38⁻ AML stem cells formed the basis for their selective elimination by echinomycin. To test if this was also the case for TP53-mutated AML, we compared mRNA levels of HIF-1α target genes among sorted CD34⁺CD38⁻, CD34″CD38⁻, CD34″CD38⁺, or CD34⁺CD38⁺ subsets for four clinical AML samples: AML 277 and AML 172 (TP53-mutated) and AML 71 and AML 132 (TP53-wild type), and the primer sequences are listed in Table 3.

TABLE 3
The primer sequences for qPCR

| | |
|---|---|
| hSLC2A3-real-F | CTTGAAGACTTGAATTAGATTACA |
| | (SEQ ID NO. 1) |
| hSLC2A3-real-R | TGTTGTAGCCAAAATTGGAAAGAGC |
| | (SEQ ID NO. 2) |
| hcMYC-real-F | TGGTGCTCCATGAGGAGAACCGC |
| | (SEQ ID NO. 3) |
| hcMYC-real-R | GCCAGGAGCCTGCCTCTTCCAC |
| | (SEQ ID NO. 4) |
| hL37-real-F | TGTGGCTACCCTGCCAAGCGCAAGAG |
| | (SEQ ID NO. 5) |
| hL37-real-R | ACAGCTGCCCTCTTGGGTTT |
| | (SEQ ID NO. 6) |

Consistent with the bioinformatics data presented in Table 1 and FIG. 1A, our quantitative-PCR (qPCR) analysis revealed that levels of HIF-1α targets GLUT, cMYC and VEGF were altogether higher in TP53-mutated samples vs—wild type samples, regardless of the population analyzed. Notably, however, the expression of these targets was significantly higher in the CD34⁺CD38⁻ stem cell subsets compared to the remaining fractions, regardless of TP53 mutational status (FIG. 1G). Taken together, the data indicated that HIF-1α plays a critical role in the CD34⁺CD38⁻ stem cell subset of TP53-mutated AML, and that the elevated HIF-1α activity compared to other subsets underlies the sensitivity of these cells to echinomycin. Importantly, the data suggested that targeting HIF-1α by echinomycin might offer a therapeutic advantage in TP53-mutated AML in vivo, which is generally refractory to standard chemotherapies.

Methods

Mice

Male and female Nod.Scid.Il2rg⁰ (NSG) mice aged 6-8 weeks were purchased from the Jackson Laboratory.

AML Samples

We used World Health Organization (WHO) AML diagnostic criteria (>20% myeloblasts in the bone marrow or peripheral blood) and determined WHO subclassification through review of data from the time of diagnosis. The clinical characteristics of patients with AML are listed in Table 4. THP1 cells were purchased from ATCC and tested negative for mycoplasma contamination.

TABLE 4
Mutation Sites and Percentage of TP53 in Clinical samples of Patients with TP53-mutated AML

| | Case No. | Age | Sex | Diagnosis | Treated/untreated | WHO | Karyotype |
|---|---|---|---|---|---|---|---|
| 1 | PB-171 | 64 | F | AML | Untreated | AML with myelo- | 44-45, XX, add(1)(p36.1), −5, |
| | | | | | | dysplasia- | add(12)(p13), |
| | | | | | | associated | der(14)t(1; 14)(p22; p13), −16, |
| | | | | | | changes | −17, −22, |
| | | | | | | | +1-3mar[cp19]/ |
| 2 | PB-172 | 67 | F | AML | Untreated | AML with myelo- | 45, XX, inv(3)(q21q26), −5, del(7)(q22), |
| | | | | | | dysplasia- | −17, +mar[19] |
| | | | | | | associated | /46, XX[1]. |
| | | | | | | changes | |
| 3 | PB-83 | 68 | M | AML | Untreated | AML with myelo- | 39-41, X, −Y, add(Y)(q12), |
| | | | | | | dysplasia- | del(5)(q13q31), der(6)t(6; 12)(p23; q13), |
| | | | | | | associated | −7, add(12)(p12), |
| | | | | | | changes | der(10; 13)(q10; q10), −16, −18, |
| | | | | | | | add(20)(q12), −22[cp16]. |
| 4 | PB-135 | 73 | M | AML | Untreated | AML with myelo- | 58-62<3n>, XXY, add(1)(q21), −3, |
| | | | | | | dysplasia- | t(3; 16)(p21; q22), −4, −5, i(5)(p10), |
| | | | | | | associated | −7, +11, del(17)(p11.2), |
| | | | | | | changes | −18, +2-5mar[cp12]. |
| 5 | PB-253 | 63 | M | AML | Untreated | pure erythroid | 4546, XY, del(4)(q23q27), del(5)(q22q35), |
| | | | | | | leukemia | −9, psu dic(14; 21)(p11.2; p11.2), − |
| | | | | | | | 17, add(19)(q13.1), +13mar[cp11]/46, XY[9] |
| 6 | PB-277 | 47 | F | AML | Untreated | AML with myelo- | 43-45, XX, +1, add(1)(q43), |
| | | | | | | dysplasia- | del(5)(q13q31), |
| | | | | | | associated | |
| | | | | | | changes | |
| 7 | MI- | 64 | F | AML | Untreated | AML-TR | 48, XX, +8, +8, i(8)(q10)[13]/49, |
| | AML- | | | | | | idem, +i(8)(q10)[6] |
| | 012 | | | | | | |
| 8 | MI- | 55 | F | AML | Untreated | AML-MRC-2 | 45, XX, del(5)(q15q33), del(7)(q22q36), |
| | AML- | | | | | | i(11)(q10), −17[20] |
| | 147 | | | | | | |
| 9 | MI- | 69 | F | AML | Untreated | AML-MRC-1, 2 | 44-45, XX, del(5)(q22q34), −6add(6)(p23), |
| | AML- | | | | | | del(13)(q12q33), add(14)(q32), add(16)(q24), |
| | 227 | | | | | | add(17)(p11.2), |
| | | | | | | | add(20)(q11.2)[cp13][13]/43-45, sl, |
| | | | | | | | +mar [cp2][2]/45, XX, del(5)(q22q34), |
| | | | | | | | 45, XX, del(5)(q22q34)add(5)(p15), |
| | | | | | | | der(6)t(6; 17)(p24; q11.2), |
| | | | | | | | del(13)(q12q33), add(14)(q32), |
| | | | | | | | add(14*)(q32), add(16)(q24), |
| | | | | | | | del(20)(q11.2q13.3)[cp2][2]der(6)t(6; |
| | | | | | | | 17)(p24; q11.2), |
| | | | | | | | del(13)(q12q33), add(14)(q32), add(16)(q24), |
| | | | | | | | −17, del(20)(q11.2q13.3)[2]/44− |
| 10 | MI- | 48 | F | AML | Treated | AML-MRC-2, 3 | 44, XX, der(5)t(5; 7)(q35; q11.2), add(5)(q13), |
| | AML- | | | | | | −7, der(12)t (12; 13)(q21; q14), |
| | 281 | | | | | | add(13)(q11.2), |
| | | | | | | | −16, −17, −18, +2mar[3]/44, |
| | | | | | | | sl, der (7)add(7)(p13)t(7; 17)(q32; q11.2), |
| | | | | | | | der(17)t(7; 17) |
| | | | | | | | (p15; q11.2)[13])/44, XX, |
| | | | | | | | der(5)t(5; 15)(q22; q21), del(6)(q23q25), |
| | | | | | | | add(7)(p11.2), −12, |
| | | | | | | | der(13)t (12; 13)(q13; q32), −15, |
| | | | | | | | −17, add(19)(q13.3), +mar[5]. |
| 11 | MI- | 73 | F | AML | Treated | AML | 45, XX, −7[20] |
| | AML- | | | | | | |
| | 71 | | | | | | |
| 12 | MI- | 71 | M | AML | Untreated | AML | 46, XY, i(17)(q10)[13]; 45, XY, |
| | AML- | | | | | | i(17)(q10), −7[7] |
| | 132 | | | | | | |

| | | | | | Other |
|---|---|---|---|---|---|
| | TP53 1 | | TP53 2 | | mutations |
| 1 | R273C | 84% | | | TET2 48%, |
| | | | | | IDH1 49% |
| 2 | A276D | 85% | | | None |
| 3 | 695T > C | 88% | | | K-RAS |
| 4 | 857A > G | 28.80% | 724T > G | 24.70% | None |
| 5 | 733G > A | 71.50% | | | None |
| 6 | 423C > G | 51.80% | 818G > A | 44.80% | N-RAS |
| | | | | | 9.7%, |
| | | | | | N-RAS |
| | | | | | 1.5% |
| 7 | R273C | | | | None |
| 8 | R175H | | | | IDH2 |
| | | | | | (R140); |
| | | | | | FLT3 |
| | | | | | (D835) |
| 9 | Y220C | | | | None |
| 10 | | | | | None |
| 11 | Wild | | | | None |
| | type | | | | |
| 12 | Wild | | | | None |
| | type | | | | |

Cell Viability and Methylcellulose Colony Formation Assay

For the cell viability assay, AML cells (1×10⁵/well) were seeded in a 24-well plate and cultured for 24 hours prior to treatment with echinomycin at different concentrations, and then incubated for 24 or 48 additional hours. Ten μl of 10 mg/ml 3-(4,5-dimethylthiazol-2-yl)-2,5-diphenyltetrazolium bromide (MTT, Sigma) in phosphate-buffered saline was added to each well. Plates were returned to the cell culture incubator for 2 to 4 hours. The resulting MTT formazan crystals were pelleted and dissolved by adding 150 μl DMSO. Absorbance was measured at a wavelength of 490 nm. The methylcellulose colony formation assay was performed according to the manufacturer's recommendations (STEMCELL Technologies Inc., Vancouver, BC, Canada). Briefly, after 24 or 48 hour incubation with echinomycin, the aforementioned treated AML cells were washed with culture medium and seeded at 2×10⁴/well in a 24-well plate. On day 7 to 10 after AML cells were seeded, colonies were counted and analyzed by FACS. Experiments were performed in triplicate.

Flow Cytometry

Blood was collected by submandibular bleeding at different times before and after AML transplantation. Fluorochrome-labeled antibodies were directly added into whole blood. After 30 min of staining, the stained samples were treated with FACS Lysing Solution to lyse the red blood cells and then washed to remove excess antibodies and debris. Spleens were gently ground with frosted objective slides and the cells were passed through a nylon cell strainer, washed three times with RPMI 1640, labeled with antibodies and analyzed for the presence of different human cell populations. Antibodies used were FITC-conjugated mouse anti-human CD123, PE-Cy7 conjugated mouse anti-human HLA-DR, PerCP-conjugated mouse anti-human CD33, BV421-conjugated mouse anti-human CD34 and anti-human CD11b, APC-Cy7-conjugated mouse anti-human CD14 (BD Bioscience, San Jose, CA), PE-conjugated anti-human CD45, APC-conjugated anti-human CD38 (eBioscience, San Diego, CA). The stained cells were analyzed with on a BD FACS Canto II flow cytometer.

AML Xenogeneic Mouse Model

For the THP1 xenograft model, two-day old NSG pups received 1.3 Gy of irradiation prior to intrahepatic transplantation of 1×10⁶ luciferase-transduced human THP1 cells. The recipients were treated with echinomycin or vehicle by intraperitoneal injection. For TP53-mutated AML xenografts, adult NSG mice received 1.3 Gy of irradiation prior to i.v. injection with 2 to 5×10⁶ of AML cells from AML patients. Human CD45-positive (hCD45⁺) cells were monitored in the blood of recipients by FACS analysis. When the percentage of hCD45⁺ cells were detectable by flow cytometry the mice were randomized to receive different treatments. Mice that had not reconstituted human AML cells were excluded. The percentage of hCD45⁺ cells was monitored by FACS analysis to observe the therapeutic effect. For the splenocyte analysis, mice were sacrificed and splenocytes were collected and stained with antibodies specific for hCD45 and mouse leukocyte markers (mCD45) in conjunction with CD34 and CD38 or with HLA-DR, CD33, CD14, CD11b and CD123 for FACS analysis. For the animal studies, at least three mice per group were used to ensure adequate power to detect difference between groups. For all experiments testing animal survival, at least 5 animals were used per group. Animals were randomized into groups. All animal experiments have been performed at least twice. All animal studies were blinded and conducted under the guidelines of the Institutional Animal Care and Use Committee of the University of Maryland and the Children's National Medical Center.

Bioluminescence Imaging

Luciferase activity at each time point was analyzed in mice anesthetized with isoflurane 10 minutes after intraperitoneal injection of d-luciferin potassium salt (Caliper Life Sciences) at 150 mg/kg. Mice were imaged in a Xenogen IVIS Spectrum Imaging System (Caliper Life Sciences). Living Image software was used to analyze the bioluminescent image data. Total bioluminescent signal was obtained as photons/second and regions of interest were used to calculate regional signals.

PK Analysis by LC-MS/MS

NSG mice received a single intravenous dose of echinomycin at 100 ug/kg, and blood samples were collected at different time points after dosing. The plasma fraction was immediately separated and stored at −80° C. until analysis. To extract echinomycin from plasma, protein was precipitated from the plasma by mixing with acetonitrile 1:4 (v/v plasma: ACN). Echinomycin was monitored by multiple reaction monitoring (MRM) in the positive electrospray ionization mode on an ABI-5500 Qtrap (Sciex, Ontario, Canada) mass spectrometer in tandem with a Shimadzu high performance liquid chromatography (HPLC) system. The Q1 and Q3 transition of echinomycin (m/z 1101.4→1053.4) and its collision energy were selected and optimized by direct infusion. Chromatographic separation was achieved on an Agilent Poroshell 120, C18 HPLC column at a flow rate of 0.4 mL/min in 7.5 minutes by a gradient elution of water and acetonitrile containing 0.1% formic acid.

Statistical Analyses

Two tailed T tests were used to determine P values for statistical significance of all pair-wise comparisons. For kinetics studies with multiple time points, statistics were analyzed by one-way ANOVA with Tukey's multiple comparisons test. Mouse survival was estimated by Kaplan-Meier survival analysis, with statistical significance determined by the log-rank test.

### Example 2

### Echinomycin Impairs Leukemia Progression in Mice Xenografted with Primary TP53-Mutated AML

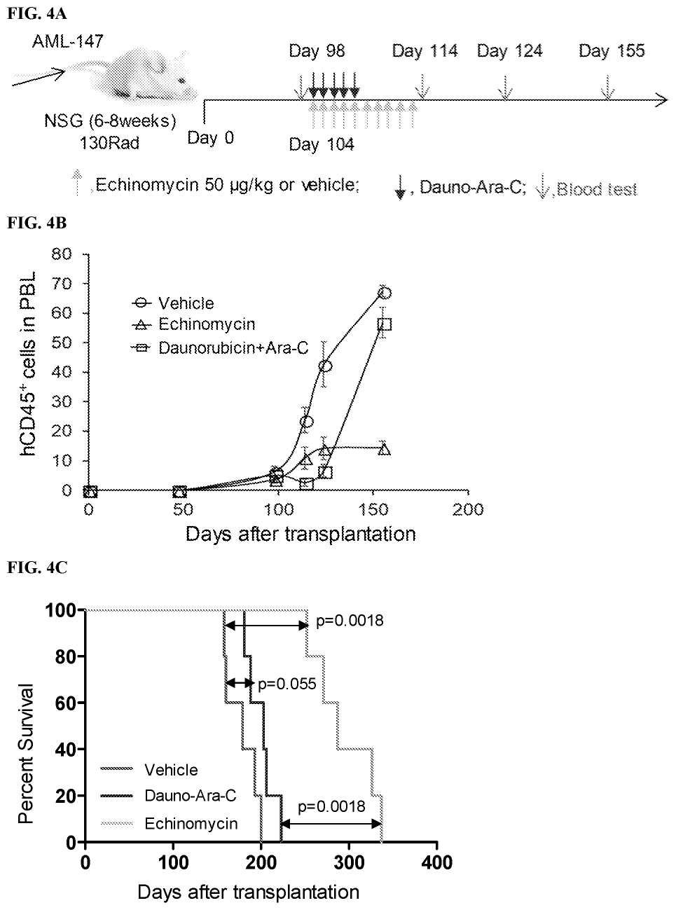

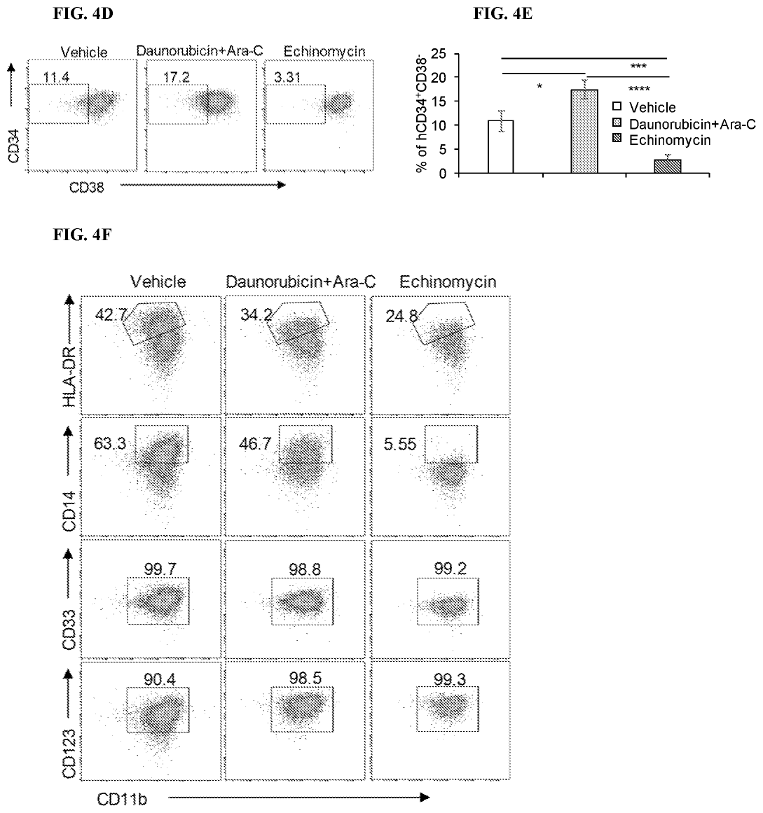

To explore the therapeutic potential of echinomycin in vivo, mice were grafted with primary human TP53-mutated AML cells (AML-147) and treated with echinomycin or conventional chemotherapy consisting of DNR+Ara-C, according to the dosing schedule in FIG. 4A. Following i.v. transplantation of 1×10⁶ TP53 mutant AML-147 cells into 1.3 Gy-irradiated NOD/SCID IL2rg^{null} (NSG) recipients, we monitored the reconstitution and progression of the cells by FACS analysis of human CD45⁺ cells in the peripheral blood. By day 98 after transplantation, between 2 to 10% of the peripheral blood cells were human CD45⁺, and we initiated treatment with Echinomycin or DNR+Ara-C on day 104. Upon completion of the treatment cycle, FACS analysis of the peripheral blood indicated that the growth of TP53-mutated AML cells was significantly inhibited in recipients either treatment. However, the leukemia cells relapsed in DNR+Ara-C-treated mice after cessation of treatment due to dose-limiting toxicity. In contrast, echinomycin-treated mice exhibited a prolonged period of growth inhibition, with peripheral blood AML blasts remaining below 20% when vehicle-treated mice had blast levels exceeding 70% and started dying (FIG. 4B). Similarly, mice treated with either drug regimen experienced significantly prolonged survival times vs vehicle-treated mice, but survival was more prolonged for echinomycin-treated mice (FIG. 4C). These data demonstrated that HIFs may serve as an effective therapeutic target for TP53-mutated AML.

Notably, while the frequencies of CD34⁺CD38⁻ AML subsets decreased in echinomycin-treated mice in comparison to that in the vehicle-treated mice, they were significantly increased in DNR+Ara-C-treated mice (FIGS. 4D-E). We also examined the frequencies of other AML cell subsets but found no significant difference between the three groups, except for a significant reduction in HLA-DR^{hi}CD11b⁺ cells in echinomycin-treated mice (FIG. 4F). Collectively, these data suggest that CD34⁺CD38⁻ AML stem cell subsets are especially sensitive to echinomycin. In contrast, DNR+Ara-C combination presumably targeted differentiated AML blasts and enriched for CD34⁺CD38⁻ AML subsets, resulting in recurrence after cessation of DNR+Ara-C treatment.

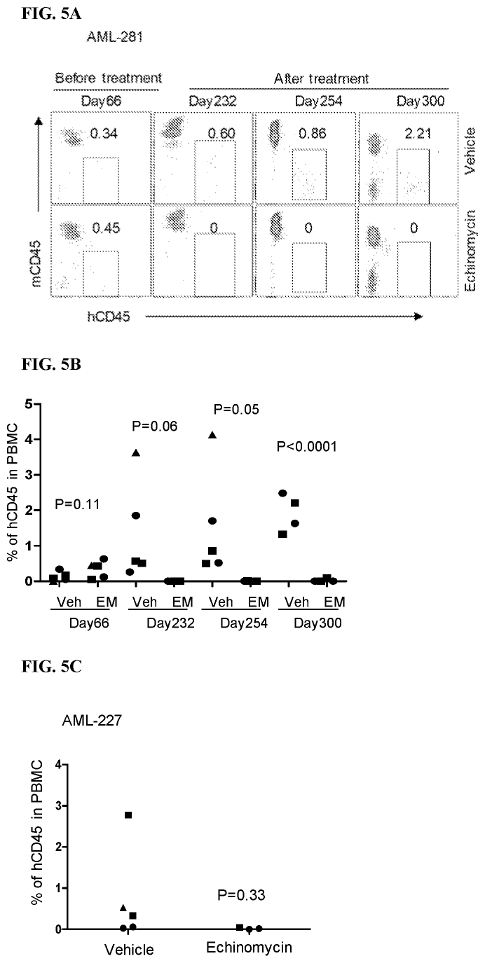

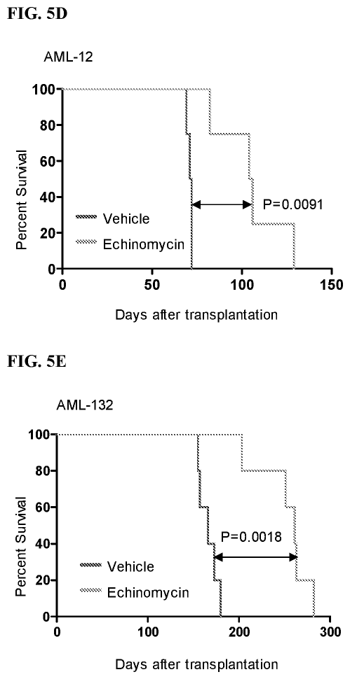

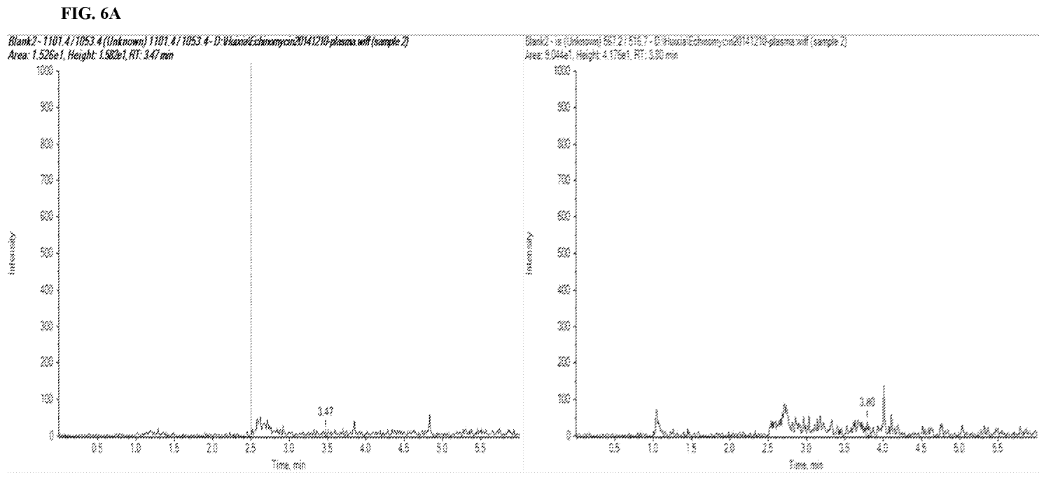

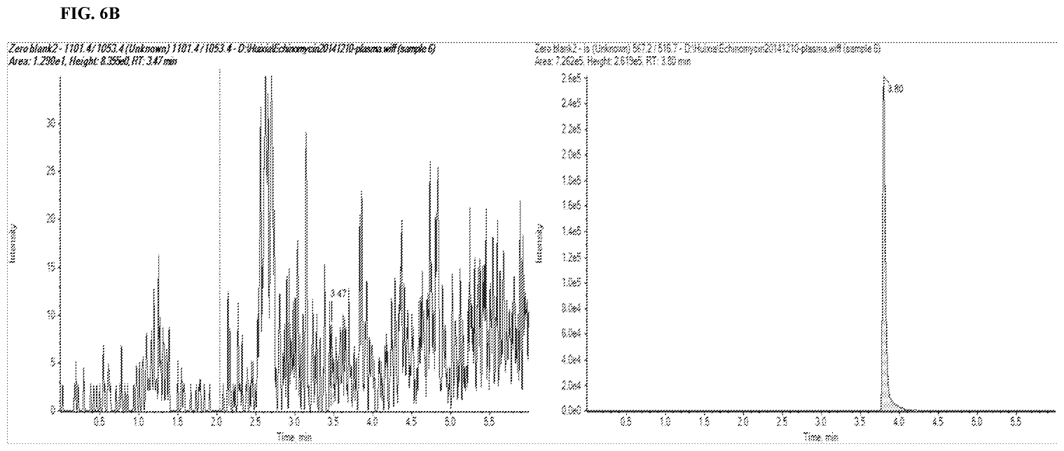

To further investigate the therapeutic effects of echinomycin in the context of TP53 mutations, we performed similar analyses in four additional xenograft models, including three primary TP53-mutated samples (AML-281, AML-227, AML-012) and one primary TP53-wild type sample (AML-132). As shown in FIGS. 5A-B, by day 66 following transplantation of AML-281 cells, approximately 0.5% human CD45⁺ cells could be detected on average in the peripheral blood of the mice. Starting on day 67, we treated the AML-281 recipients with echinomycin 10 μg/kg in a regimen consisting of three QDx5 cycles, each separated by two days of rest. FACS analysis of the peripheral blood cells showed that while the human CD45⁺ cells increased in vehicle-treated mice over the course of the study, they were undetectable in 100% of echinomycin-treated mice by day 232, and remained undetectable in nearly all of the echinomycin-treated mice until the end of the observation period on day 300 (FIGS. 5A-B). Using the same treatment regimen, we performed this experiment on mice xenografted with TP53-mutated AML-227 cells and found that human CD45⁺ cells were similarly reduced in echinomycin-treated mice compared to the vehicle group on day 300. FIG. 5C. Due to the slow growing nature of AML-281 and AML-227 primary cells in the NSG recipients, mortality was not a feasible endpoint for the studies involving these two samples. In contrast, transplantation of primary TP53-mutated AML-012 cells resulted in mortality in 100% of NSG recipients by day 75 after transplantation in the absence of therapeutic intervention; accordingly, echinomycin extended survival time in AML-012 recipients by more than 130 days (FIG. 5D). As our initial findings presented in FIG. 1 revealed elevated HIF-1α activity in TP53-mutated AML, we sought to determine if differences in TP53 genotype might correspond to an overall increase in sensitivity to echinomycin. As summarized in Table 2, in vitro treatment of AML-71 and AML-132 with echinomycin revealed modestly increased IC₅₀s for these cells when compared with multiple TP53 mutated samples, whereas the CFU indicated no difference in sensitivity to echinomycin. To test the impact of TP53 mutations on therapeutic effect of echinomycin in the xenograft models, we transplanted NSG mice with TP53-wild type AML-132 cells and observed the effects of echinomycin treatment on survival time for comparison with the TP53-mutated models. Although these cells exhibited lower levels of HIF-1α activation compared to the TP53-mutated samples, Echinomycin also significantly prolonged the survival time of the AML-132 recipients (FIG. 5E). On the basis of these findings, it is evident that TP53-mutated AML clearly exhibits higher overall HIF-1α activity compared to cases of TP53-wild type AML, although the increased activity does not necessarily equate to an obvious increase in therapeutic response to echinomycin. Rather, the results suggest that a basal requirement for HIF-1α pathway activation in the CD34⁺CD38⁻ stem cell subset, which we uncovered previously in cases of TP53-wild type AML, appears to be indispensable also in cases with TP53 mutation. Nevertheless, the finding that TP53 mutations did not abrogate therapeutic response to Echinomycin is very significant as they have rendered leukemia more resistant to conventional therapy.

Methods

As in Example 1.

### Example 3

### A Liposomal Formulation of Echinomycin Significantly Prolonged the Survival of TP53-Mutated AML Xenografts

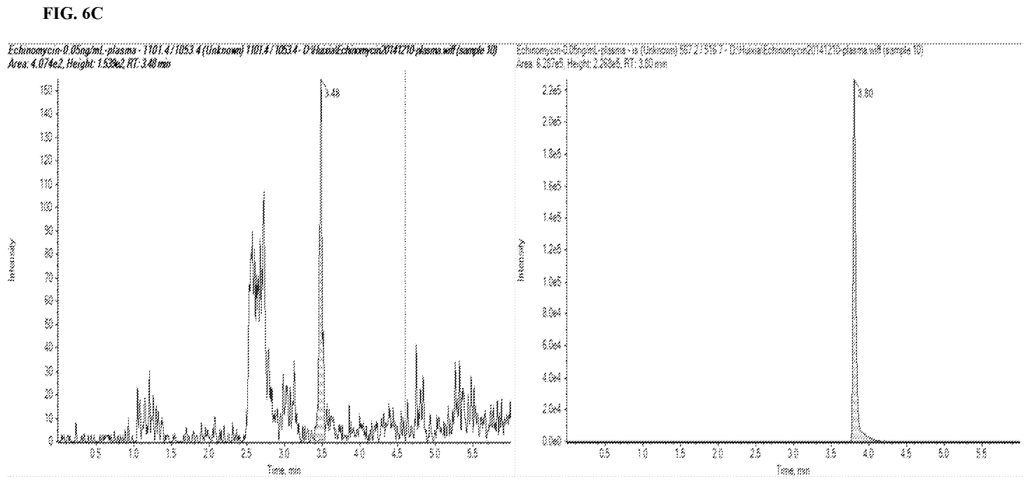

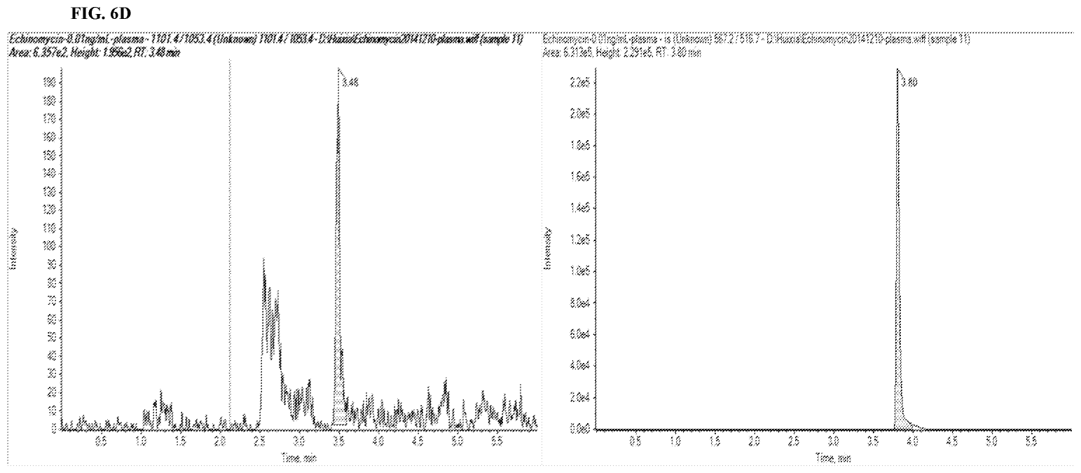

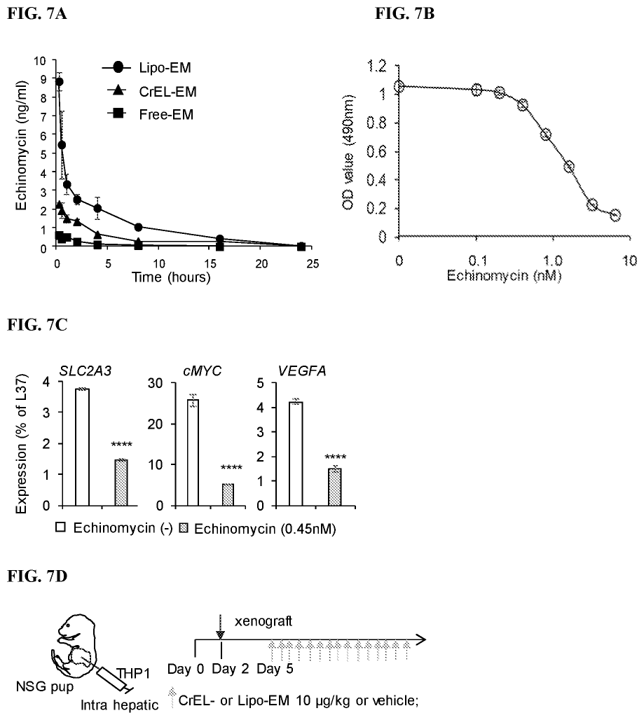

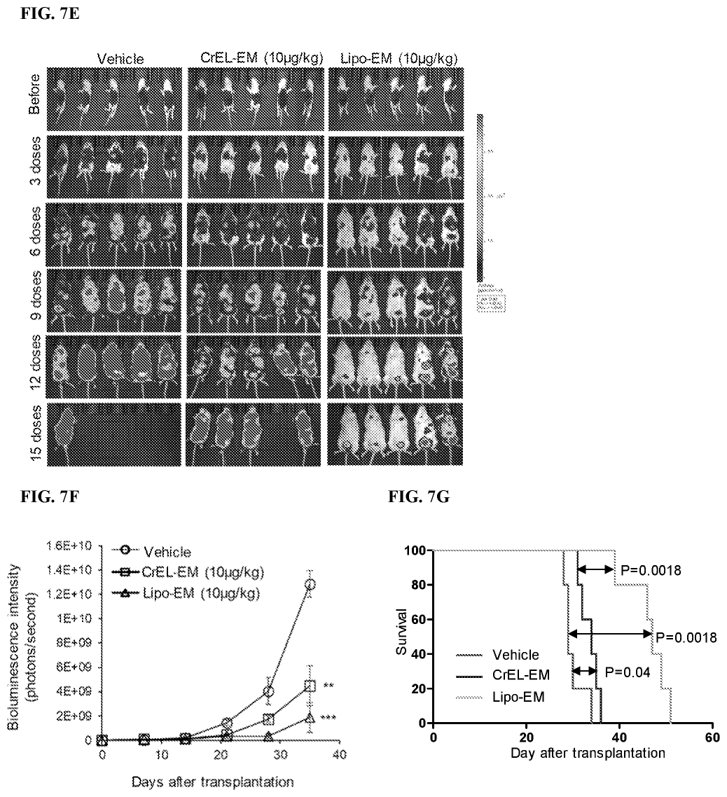

Pharmacokinetics (PK) of echinomycin in vivo have not been studied due to the lack of a sensitive analytical method for assaying echinomycin concentrations in biological matrices. To overcome this challenge, we developed and validated a sensitive LC-MS/MS method for in vivo detection of echinomycin, which we used to study the PK and tissue distribution of echinomycin in mice. As shown in FIG. 6, the new method provided a lower limit of quantification (LLOQ) for echinomycin in plasma and tissue samples of 50 pg/mL (pg/g). We studied the plasma PK of 3 echinomycin formulations following a single i.v. injection at a dose of 100 ug/kg. As shown in FIG. 7A, free echinomycin, consisting of DMSO/saline (1:9, v/v), reached plasma concentrations of 0.61, 0.51, 0.11 and 0.063 ng/ml at 0.25, 1, 4 and 8 hrs following administration, respectively. At the same respective time points, plasma echinomycin concentrations reached 2.3, 1.5, 0.64 and 0.28 ng/ml when formulated in Cremophor EL/ethanol/saline (1:1:18, v/v) (CrEL-EM), and 8.9, 3.0, 2.1 and 1.0 ng/ml when formulated in liposomes consisting of HSPC:Cholesterol:DSPE-mPEG2000 (57:38:5, mol:mol) at a 3% drug/lipid molar ratio. The results demonstrated that formulating echinomycin in PEGylated liposomes significantly prolonged the circulation time in the bloodstream compared to the alternative formulations. Table 5 shows the mean values for the pharmacokinetic parameters in plasma after single doses of the three formulations of echinomycin administered to mice. Echinomycin exposure for the liposomal formulation was much higher than for free drug and CrEL-EM. The estimated AUCₗₐₛₜ of the liposomal formulation was 13.6- and 3.19-fold that of free drug and CrEL-EM, respectively. For echinomycin Cₘₐₓ in mouse plasma, the liposomal formulation was 13.2 and 3.90 times higher than for free drug and CrEL-EM. In addition, liposomal formulation and CrEL-EM showed a similar elimination half-life (T_{1/2}) which was around twice as long as for free drug. Taken together, the liposomal formulation showed the best pharmacokinetic behavior in mice among these three formulations.

TABLE 5
PK parameters (Mean ± SD) of Echinomycin in mouse plasma
after administration of 0.1 mg of three formulation of Echinomycin.

| | | | AUCₗₐₛₜ |
|---|---|---|---|
| | T_{1/2} (hr) | Cₘₐₓ (ng/mL) | (hr*ng/mL) |
| Free-EM | 2.46 ± 0.29 | 0.67 ± 0.24 | 1.78 ± 0.55 |
| CrEL-EM | 4.68 ± 0.65 | 2.27 ± 0.11 | 7.58 ± 0.45 |
| | (P = 0.0057)^{a} | (P = 0.00047) | (P = 0.00015) |
| Lipo-EM | 4.28 ± 0.61 | 8.85 ± 0.88 | 24.17 ± 2.58 |
| | (P = 0.0095) | (P = 0.0001) | (P = 0.00012) |

^{a}P value to Free-EM.

Methods

As in Example 1.

### Example 4

### Echinomycin Inhibits Expansion of Human TP53-Null THP1 Cells in Xenografted Mouse Model

THP1 is an acute monocytic leukemia cell line that has a homozygous 26-base deletion starting at codon 174 of the TP53 coding sequence. We first investigated the therapeutic effect of echinomycin on TP53-null THP1 cells in vitro. THP1 cells were incubated for 48 hrs with 0.1-6.4 nM echinomycin and cell viability was determined by MTT assay (FIG. 7B). The THP1 cells exhibited a concentration-dependent decrease in cell viability in response to echinomycin treatment, with an EC50 of 1.25 nM (FIG. 7B) as well as suppression of HIF-1α target genes (FIG. 7C).

To evaluate the therapeutic effect of liposomal echinomycin against TP53-mutated AML cells, we transplanted THP1 into newborn NSG pups via intrahepatic injection (FIG. 7D). Mice were treated with 10 μg/kg of echinomycin formulated in either CrEL or liposomes on a QDx15 schedule beginning on day 3 after THP1 cell transplantation (FIG. 7D). Mice were imaged on day 3 after THP1 cell transplantation (before) and throughout the treatment cycle. In vehicle-treated mice, the average bioluminescence signal intensity increased over the course of three weeks after transplantation, indicating progressive leukemia growth, and all vehicle-treated mice died from leukemia within 35 days (FIGS. 7E-G). In contrast, mice treated with echinomycin displayed reduced leukemia growth, indicating a therapeutic effect of echinomycin. CrEL-EM modestly inhibited bioluminescence signal intensity and provided a marginal, albeit significant, improvement in survival time; on the other hand, the bioluminescence signal barely increased during the same time period in mice treated with liposomal echinomycin, and these mice survived for more than 50 days (FIGS. 7E-G). The survival of recipients of liposomal echinomycin was significantly longer vs mice that received vehicle or CrEL-EM (FIG. 7G). These results indicated a robust therapeutic effect of liposomal echinomycin against THP1 leukemia cells in vivo and demonstrate a significant anti-leukemia effect of liposomal echinomycin in TP53-null leukemia cells.

Methods

As in Example 1.

### Example 5

### Liposomal Echinomycin Suppressed the Growth of Human Patient-Derived Xenograft TP53-Mutated AML Cells in Xenograft Mouse Model

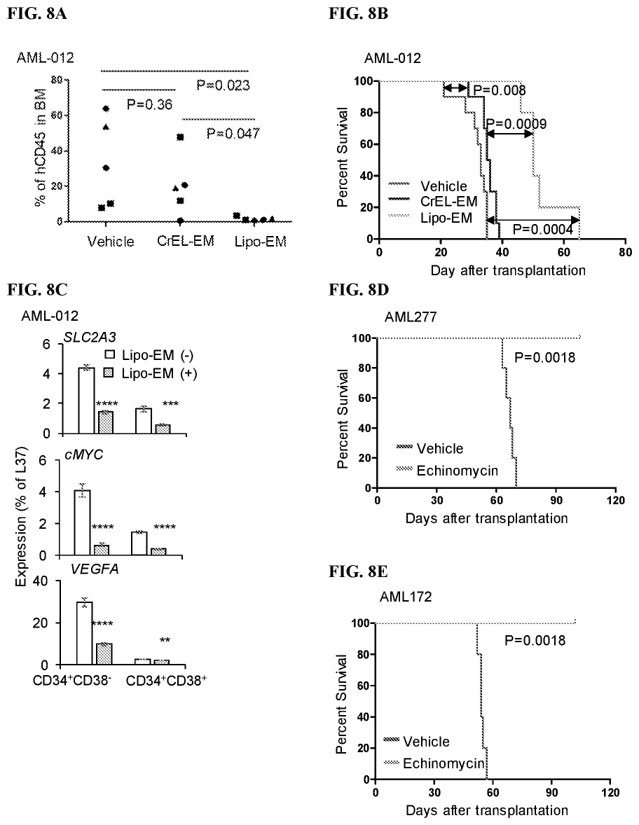

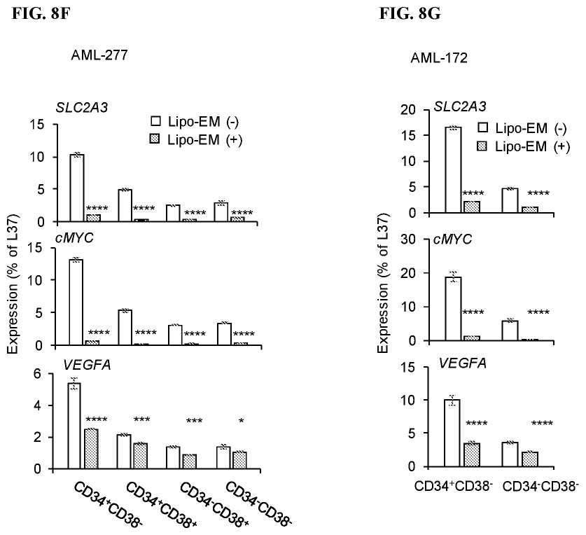

To further examine the therapeutic effect of liposomal echinomycin using patient-derived AML cells, we transplanted NSG mice with AML-012 and treated the mice with either CrEL or liposomal echinomycin, Q3D×5 starting day 10 after transplantation to observe the effects of treatment using different formulations. Unlike other AML cells we used, AML-012 could not be detected in peripheral blood throughout the study period. Therefore, to observe the effects of the echinomycin treatment on the AML-012 blast growth at a fixed time point, we sacrificed 5 mice from each group on day 30 and analyzed human blasts in the BM. Liposomal echinomycin, but not CrEL-echinomycin, suppressed AML-012 blasts in the BM in this xenograft model (FIG. 8A). The survival of remaining mice in each group was significantly longer vs mice that received vehicle or CrEL-EM (FIG. 8B). To observe the pharmacodynamic activity of liposomal echinomycin on the HIF-1α pathway among different AML subsets, we examined levels of HIF-1α target genes in the sorted human CD34⁺CD38⁻ or CD34⁺CD38⁺ AML-012 blasts isolated from BM of xenograft recipients of either vehicle or liposomal echinomycin. As expected, the levels of HIF-1α target genes were significantly reduced in the AML-012 blasts, regardless of the cell subset analyzed, although the target gene suppression was generally more profound among the CD34⁺CD38⁻ cells (FIG. 8C). We performed similar experiments in additional TP53-mutated AML primary cells. As shown in FIGS. 8D and E, liposomal echinomycin treatment was effective in extending the survival time of mice xenografted with AML-277 or AML-172 primary cells, respectively. Accordingly, HIF-1α target genes were also reduced in the sorted AML blasts isolated from the BM of liposomal echinomycin-vs vehicle treated mice in both of these models, and the suppression was similarly most profound in the CD34⁺CD38⁻ subsets (FIG. 8F, G). Altogether, these results further indicated a robust therapeutic effect of liposomal echinomycin against patient-derived leukemia cells in vivo and demonstrate a significant anti-leukemia effect of liposomal echinomycin in TP53-mutated human leukemia cells.

Methods

As in Example 1.

1. A method for treating acute myeloid leukemia (AML) in a mammal in need thereof, wherein the AML is TP53-mutated AML, the method comprising administering to the mammal a Hypoxia-Inducible Factor (HIF) inhibitor.

2. The method of claim 1, wherein the HIF inhibitor is a HIF1α inhibitor.

3. The method of claim 2, wherein the HIF inhibitor is selected from the group consisting of echinomycin, 2-methoxyestradiol, and geldanamycin.

4. The method of claim 1, wherein the echinomycin is administered at a non-toxic dose.

5. The method of claim 4, wherein the dose is 1-1000 μg/m².

6. The method of claim 1, wherein the mammal is a human.

7. The method of claim 1, wherein the TP53-mutated AML is characterized by enriched activity of one or more HIF1A target genes, as compared to one or more wild-type TP53 AML samples.

8. The method of claim 7, wherein the one or more HIF1A target genes are selected from the group consisting of TFRC, CMYC, HK1, SLC2A1, SNAI1, ALDOC, CP, TF, GLUT, and VEGF.

9. The method of claim 1, wherein the TP53-mutated AML is refractory to standard therapy.

10. The method of claim 9, wherein the standard therapy comprises administration of daunorubicin and cytarabine (DNR+Ara-C) to the mammal.

11. A pharmaceutical composition comprising a HIF inhibitor formulated in PEGylated liposomes.

12. The pharmaceutical composition of claim 11, wherein the PEGylated liposomes comprise one or more of hydrogenated soy phosphatidylcholine (HSPC), cholesterol, and distearoylphophatidylethanolamine (DSPE)-mPEG2000.

13. The pharmaceutical composition of claim 12, wherein the HSPC, cholesterol, and DSPE-mPEG2000 are present in the PEGylated liposomes at molar ratios of 30-40%, and 1-5%, respectively.

14. The pharmaceutical composition of claim 13, wherein the PEGylated liposomes comprise, as molar ratios, about 57% HSPC, about 38% cholesterol, and about 5% DSPE-mPEG2000.

15. The pharmaceutical composition of claim 14, comprising the HIF inhibitor and PEGylated liposomes at a molar ratio of 3% drug/lipid.

16. The pharmaceutical composition of claim 15, wherein the HIF inhibitor is echinomycin.

17. A formulation of echinomycin comprising: PEGylated liposomes comprising, as molar ratios, about 57% HSPC, about 38% cholesterol, and about 5% DSPE-mPEG2000; and echinomycin; wherein echinomycin and the PEGylated liposomes are present at a molar ratio of 3% drug/lipid.

18. A method of treating TP53-mutated AML in a human in need thereof, the method comprising administering to the human the formulation of claim 17.

19. The method of claim 18, wherein the TP53-mutated AML is refractory to standard therapy.

20. The method of claim 19, wherein the standard therapy comprises administration of daunorubicin and cytarabine (DNR+Ara-C) to the human.

21.-46. (canceled)
